(19) United States
(12) Patent Application Publication
(10) Pub. No.: US 2015/0355191 A1
(43) Pub. Date: Dec. 10, 2015

(54) Cell-Based Assay for Neutralizing Antibodies

(71) Applicant: BIOMIMETIC THERAPEUTICS, LLC, Franklin, TN (US)

(72) Inventors: Leo B. Snel, Nashville, TN (US); Luis Solchaga, Franklin, TN (US)

(73) Assignee: BioMimetic Therapeutics, LLC, Franklin, TN (US)

(21) Appl. No.: 14/425,025

(22) PCT Filed: Jun. 28, 2013
(86) PCT No.: PCT/US13/48564

§ 371 Date: Feb. 28, 2015

Related U.S. Application Data
(60) Provisional application No. 61/670,390, filed on Jul. 11, 2012.

(51) Int. Cl. G01N 33/68; G01N 33/74

(52) U.S. Cl. CPC G01N 33/6854; G01N 33/74; G01N 2333/49; G01N 2333/71

(57) ABSTRACT

The present disclosure relates to a method for detecting the presence of PDGF neutralizing antibodies in a serum sample, comprising contacting a population of cells with i) a serum sample, and ii) PDGF, wherein the cells comprise a PDGF receptor; and detecting an amount of a biomarker in the population of cells, wherein the biomarker indicates binding of the PDGF with the PDGF receptor. The present disclosure also relates to a method of determining the presence of PDGF neutralizing antibodies in a subject who has received or is currently receiving a treatment comprising PDGF.

### TECHNICAL FIELD

The present disclosure relates to a method for detecting the presence of protein therapeutic-neutralizing antibodies, such as PDGF-neutralizing antibodies, in a serum sample.

### BACKGROUND

Tissue repair occurs as a result of a complex series of events. For successful tissue repair to take place, the appropriate cell types must be recruited to the site of injury. One of the proteins involved in triggering this process is platelet-derived growth factor (PDGF), which stimulates a wide spectrum of biological activities that places it at the top of the natural wound-healing cascade. PDGF is responsible for stimulating a variety of cellular events needed for the initiation and progression of tissue repair. PDGF is released from platelets at the site of injury and has a localized stimulatory effect on the wound-healing process. PDGF is a cationic, heat stable protein found in a variety of cell types, including the granules of circulating platelets, vascular smooth muscle cells, endothelial cells, macrophage, and keratinocytes, and is known to stimulate in vitro protein synthesis and collagen production by fibroblasts. It is also known to act as an in vitro mitogen and chemotactic agent for fibroblasts, smooth muscle cells, osteoblasts, and glial cells.

The PDGF family consists of PDGF-A, -B, -C, and -D, comprises five different members that are found naturally in the body, PDGF-AA, PDGF-AB, PDGF-BB, PDGF-CC and PDGF-DD; the most abundant member is the AB dimer isoform. The BB isoform of PDGF is a homodimer of two antiparallel B-chains that are covalently linked through disulfide bonds. Recombinant human PDGF-BB (rhPDGF-BB) can be manufactured using recombinant DNA technology in a yeast expression system. The gene that codes for the human sequence of the PDGF B-chain is inserted into yeast cells (Saccharomyces cerevisiae) and then activated to cause the production of the PDGF B-chain protein. The correctly folded mature protein is secreted from the yeast cell into the culture medium, and subsequently purified from the media by several chromatographic processes. The highly purified rhPDGF-BB can be formulated in 20 mM sodium acetate, pH 6.0, and contains less than 1% high molecular weight species.

rhPDGF-BB has been shown to stimulate wound healing and bone regeneration in both animals and humans. It is approved in both the United States and Europe for human use in topical applications to accelerate healing of chronic diabetic foot sores. rhPDGF-BB has also been shown to be effective either singly or in combination with other growth factors for improving periodontal regeneration, i.e., regrowth of bone, cementum, and ligament around teeth (see, e.g., U.S. Pat. No. 5,124,316, incorporated herein by reference).

There are two structurally related PDGF receptors: PDGF Rα and PDGF Rβ. These receptors are independently regulated, but have been found to be expressed together on fibroblasts, smooth muscle cells and neurons. Other cell types, such as platelets and rat liver endothelial cells express only PDGF Rα, while mouse capillary endothelial cells express only PDGF Rβ. The receptors have roughly equivalent binding for PDGF-BB. Binding of PDGF-BB induces the formation of homodimers and/or heterodimers of the receptors (Heldin and Westermark). Depending on the number and ratio of the receptors present on a cell, the cell will be more or less responsive to the different PDGF family members. rhPDGF-BB has the ability to bind with high affinity to both receptors, providing it with unique properties within the PDGF family.

PDGF receptors are members of the receptor-tyrosine kinase family and have intrinsic kinase activity upon ligand-induced dimerization have intrinsic kinase activity, which results in autophosphorylation. Phosphorylation of the PDGF receptors is a highly specific activity of PDGF. The phosphorylated receptors act as a docking site for kinases, phosphatases, and adaptor molecules. For example, the tyrosine at position 751 of human PDGF Rβ has been shown to be a docking site for phosphinositide 3-kinase (Kazluskas and Cooper, 1990), which has been shown to be involved in PDGF-BB induced cell proliferation and migration (Bornfelt et al., 1995). Thus, the phosphorylation of the PDGF receptors is followed by a cascade of intracellular signal transduction that ultimately results in cell activities such as mitosis or migration.

Mechanisms which facilitate clearance of PDGF, which limit the systemic availability and regulate the activity of PDGF at the local injury site, have been identified. PDGF is rapidly cleared from circulation, with a reported systemic half-life of approximately two minutes, as measured following intravenous administration (Bowen-Pope et al., 1984). The mechanism for clearance of PDGF from systemic circulation has been characterized in a number of studies. The presence of a plasma-binding protein for PDGF was first described by Bowen-Pope et al. and Raines et al. (1984), and was shown to inhibit the biological activity of the bound PDGF. Characterization of the interaction of PDGF to plasma binding proteins determined that α2-macroglobulin is the protein responsible for plasma binding (Raines et al, 1984; Huang et al., 1984).

All of this evidence supports the conclusion that a single, local administration of rhPDGF-BB exhibits pharmacologic action at the site of delivery, and that the rhPDGF-BB released from the implantation site is sequestered by endogenous mechanisms (α₂-macroglobulin) and cleared rapidly from systemic circulation, thereby preventing high systemic exposure.

Nevertheless, as with any human protein therapeutic, there is the potential for the development of anti-protein antibodies in patients receiving rhPDGF-BB. In some cases, these antibodies could exhibit neutralizing activity. Such PDGF-neutralizing antibodies, if present, would prevent the binding of PDGF to its receptor and prevent the cell signaling necessary for cell activation. These, PDGF-neutralizing antibodies, if have present, have the potential to nullify not only the therapeutic effect of exogenously administered rhPDGF-BB, but also the normal activity of endogenous PDGF. Similarly, other growth factors that may be used therapeutically also could exhibit neutralizing anti bodies, such as bone morphogenic proteins (BMPs), epidermal growth factor (EGF), fibroblast growth factor (FGF), insulin-like growth factor (IGF), transforming growth factor-α (TGF-α), transforming growth factor-β(TGFβ), tumor necrosis factor-α (TNF-α), and vascular endothelial growth factor (VEGF).

Accordingly, there is a need for methods of analyzing serum samples of subjects receiving growth factor-containing therapeutics, such as PDGF, for the presence of antibodies capable of not only binding to, but also neutralizing the biologic activity of the growth factor.

### BRIEF SUMMARY

One aspect of the disclosure relates to a method for detecting the presence of protein therapeutic neutralizing antibodies in a serum sample, comprising: contacting a population of cells with: i) a serum sample that may contain the protein therapeutic neutralizing antibodies, and the protein therapeutic, wherein the cells comprise a receptor for the protein therapeutic; and detecting a biomarker indicative of binding of the protein therapeutic with the receptor. More particularly, the present disclosure provides a method for detecting the presence of growth factor neutralizing antibodies in a serum sample, comprising: contacting a population of cells with i) a serum sample, and the growth factor, wherein the cells comprise a growth factor receptor; and detecting an amount of a biomarker in the population of cells, wherein the biomarker is indicative of binding of the growth factor to the growth factor receptor, and correlating the amount of the biomarker with the presence of the growth factor neutralizing antibodies.

In some embodiments, the growth factor is selected from the group consisting of platelet-derived growth factor (PDGF), bone morphogenic proteins (BMPs), epidermal growth factor (EGF), fibroblast growth factor (FGF), insulin-like growth factor (IGF), transforming growth factor-α (TGF-α), transforming growth factor-β (TGF-β), tumor necrosis factor-α (TNF-α), and vascular endothelial growth factor (VEGF). In some embodiments, the growth factor is PDGF, the neutralizing antibodies are PDGF neutralizing antibodies, and the cells comprise a PDGF receptor. The PDGF receptor can be PDGF Rα, PDGF Rβ, or a mixture thereof. In certain embodiments, the PDGF receptor is PDGF Rβ.

The PDGF can be selected from the group consisting of PDGF-AA, PDGF-BB, PDGF-AB, PDGF-CC, PDGF-DD and combinations thereof. In certain embodiments, the PDGF is PDGF-BB. In other embodiments, the PDGF is recombinant human PDGF, such as rhPDGF-AA, rhPDGF-BB, rhPDGF-AB, rhPDGF-CC, rhPDGF-DD and combinations thereof.

The PDGF may be present at a concentration effective for binding to the PDGF receptor and inducing formation of the biomarker, for example a concentration ranging from about 0.5 ng/mL to about 50 μg/mL. In other embodiments, the PDGF has a concentration ranging from about 0.5 ng/mL to about 100 ng/mL, about 1 ng/mL to about 50 ng/mL, about 1 ng/mL to about 20 ng/mL, or about 1 ng/mL to about 10 ng/mL. In other embodiments, the concentration of PDGF may be about 2.5 ng/mL, about 5 ng/mL, or about 10 ng/mL. In still other embodiments, the PDGF has a concentration ranging from about 0.01 μg/mL to about 50 μg/mL. For example, the concentration of PDGF may be in a range of about 0.1 μg/mL to about 50 μg/mL, or about 0.1 μg/mL to about 10 μg/mL. In other embodiments, the PDGF has a concentration of about 0.1 μg/mL to about 5 μg/mL, about 1 μg/mL to about 5 μg/mL, about 1 μg/mL, about 1.16 μg/mL, about 5 μg/mL or about 45 μg/mL. It is to be understood the aforementioned concentrations are merely examples of particular embodiments, and that the concentration of PDGF may be within any of the concentration ranges recited above.

In some embodiments, the biomarker is a phosphorylated growth factor receptor, such as a phosphorylated PDGF receptor. Phosphorylation of the PDGF receptor is highly specific to PDGF. Thus, measurement of phosphorylated PDGF, instead of other downstream effects of PDGF signaling such as cell proliferation, reduces any effects of other bioactive molecules that may be present in the serum sample. In certain embodiments, the phosphorylated PDGF receptor is phosphorylated PDGF Rβ.

The biomarker, such as a phosphorylated PDGF receptor, can be detected using any method known in the art. For example, the biomarker can be detected using Western Blot analysis or by using an ELISA assay. When an ELISA assay is used to detect phosphorylated PDGF Rβ, the optical density can be measured to detect the phosphorylated PDGF Rβ. In certain embodiments, a cut point can be calculated to determine when PDGF neutralizing antibodies are present in the sample. In certain embodiments, a floating cut point is used. Thus, the present methods can determine if PDGF neutralizing antibodies are or are not present in the serum sample in an amount sufficient to significantly neutralize PDGF.

In some embodiments, the contacting step comprises incubating the cells in the serum sample and the PDGF. The contacting step may be performed using a suspension of the cells, or the cells may be adhered to culture plates during the contacting step.

The cells may be lysed prior to the detecting step. In some embodiments, the cells are detached from the culture surface prior to lysing the cells. In other embodiments, the cells are lysed while still attached to cell culture surface.

The population of cells used in the present methods can be human cells. For example, the cells can be human neonatal fibroblast cells or MG-63 osteosarcoma cells.

The serum sample that may contain or is suspected of containing PDGF-neutralizing antibodies is, in some embodiments, obtained from a subject who has received or is currently receiving a treatment comprising PDGF. In some embodiments, the treatment comprises PDGF-BB, such as rhPDGF-BB. Thus, in certain embodiments, the PDGF-neutralizing antibodies are PDGF-BB neutralizing antibodies.

In some embodiments, a floating cut point can be determined for comparative purposes. For example, the method may further comprise determining a floating cut point based on a negative base pool, correlating the floating cut point with the presence of growth factor neutralizing antibodies, and comparing the amount of the biomarker in the population of cells to the floating cut point. Thus, a floating cut point allows the determination of the presence of neutralizing antibodies and the determination of acceptable levels of neutralizing antibodies.

The present methods can advantageously be used to monitor subjects receiving treatments comprising a growth factor for the presence of-neutralizing antibodies. Accordingly, another embodiment of the present disclosure relates to a method of determining the presence of PDGF neutralizing antibodies in a subject, comprising: providing a serum sample from the subject, contacting a population of cells with: i) a serum sample that may contain the PDGF neutralizing antibodies, and ii) PDGF, wherein the cells comprise a PDGF receptor; and detecting an amount of a biomarker in the population of cells, wherein the biomarker indicates binding of the PDGF with the PDGF receptor. When PDGF neutralizing antibodies are detected in quantities sufficient to interfere with PDGF signaling, then the present method, in certain embodiments, comprises ending the treatment comprising PDGF. In certain embodiments, the disclosure provides a method of treating a subject comprising administering a therapeutic comprising PDGF to the subject, determining the presence of PDGF neutralizing antibodies in the subject, and i) continuing treatment if the PDGF neutralizing antibodies are not present in an amount sufficient to interfere with PDGF signaling, or ii) stopping treating if PDGF neutralizing antibodies are present in an amount sufficient to interfere with PDGF signaling.

### BRIEF DESCRIPTION OF THE DRAWINGS

FIGS. 1A and 1B depict Western Blots for PDGF Rβ phosphorylated at Y751. After rhPDGF-BB stimulation, cells were lysed and proteins separated on a 5% Tris-HCl gel and transferred to a PDVF membrane. Western blotting using rabbit anti-phospho-PDGF Rβ (Y751) antibody was performed. FIG. 1A: depicts a Western blot of human neonatal dermal fibroblasts that have been unstimulated or stimulated with rhPDGF-BB at 45 μg/mL for 2, 5, 10 or 30 minutes. FIG. 1B: depicts a Western blot of human neonatal dermal fibroblasts that have been unstimulated or stimulated for 2 minutes with rhPDGF-BB at 45, 5.65, 0.7, 0.09, or 0.01 μg/mL.

FIG. 2 depicts Western blot detection of PDGF Rβ phosphorylated at Y751. Human neonatal dermal fibroblasts were incubated for 2 minutes in PBS or NBP at 1:20, 1:50 or 1:100 with and without 1.16 μg/mL of rhPDGF-BB. Cells were lysed and proteins separated on a 5% Tris-HCl gel, transferred to a PDVF membrane, then probed with a rabbit anti-phospo-PDGF Rβ (Y751) antibody.

FIG. 3 depicts ELISA detection of PDGF R Rβ phosphorylated at Y751. Human neonatal dermal fibroblasts were incubated for 2 minutes in PBS or NBP at 1:20, 1:50, or 1:100 with and without 1.16 μg/mL of rhPDGF-BB. Cells were lysed and lysates analyzed with a sandwich ELISA, capturing human PDGF Rβ and detecting phosphorylated PDGF Rβ, and the concentration of phosphorylated PDGF RB for each lysate was calculated using the standard curve.

FIG. 4 depicts ELISA detection of PDGF Rβ phosphorylated at Y751. Human MG-63 osteosarcoma cells were incubated for 2 minutes in PBS or NBP at 1:20 with and without 1.5 μg/mL of rhPDGF-BB; rhPDGF-BB samples were pre-incubated with the GαBB antibody at different concentrations for 1 hour prior to stimulation of the cell suspensions. Cells were lysed and the lysates analyzed with a sandwich ELISA detecting phosphorylated PDGF Rβ. Three independent replicate sets of lysates were prepared by the same analyst in three different dates.

FIG. 5 depicts ELISA detection of PDGF Rβ phosphorylated at Y751. Human MG-63 osteosarcoma cells were incubated for 2 minutes in PBS or NBP at 1:20 with and without 1.5 μg/mL of rhPDGF-BB: rhPDGF-BB samples were pre-incubated with the GαBB antibody at different concentrations for 1 hour prior to stimulation of the cell suspensions. Cells were lysed and lysates analyzed with a sandwich ELISA detecting phosphorylated PDGF Rβ. Two independent replicate sets of lysates were prepared by two analysts on the same date.

FIG. 6 depicts ELISA detection of PDGF Rβ phosphorylated at Y751. Human MG-63 osteosarcoma cells were incubated for 5 minutes in assay medium (AM) or assay medium supplemented with 5% normal human serum (5) with different concentrations of rhPDGF. Cells were lysed and lysates analyzed with a sandwich ELISA detecting phosphorylated PDGF Rβ. The cells remain attached to the cell surface during stimulation with PDGF and during lysis of the cells. Two different cell densities were used. A: 10⁶ cells/well. B: 2.5×10⁶ cells/well.

FIG. 7 depicts ELISA detection of PDGFβ. Human MG-63 osteosarcoma cells seeded at either 4×10⁴ (400) or 2×10⁴ (200) cells/cm² were incubated for 5, 10, 15 or 30 minutes in assay medium supplemented with 5% normal human serum (control) or with assay medium supplemented with 5% normal human serum and 10 ng/mL rhPDGF-BB. Cells were lysed and lysates analyzed with a sandwich ELISA detecting Total PDGF Rβ.

FIG. 8 depicts ELISA detection of PDGF Rβ phosphorylated at Y751. Human MG-63 osteosarcoma cells were incubated for 5, 10, 15 or 30 minutes in assay medium supplemented with 5% normal human serum (C) or with assay medium supplemented with 5% normal human serum and 10 ng/mL rhPDGF-BB (T). The experiment was performed twice using two different lots of human serum (1 and 2). Cells were lysed and lysates analyzed with a sandwich ELISA detecting phosphorylated PDGF Rβ.

FIG. 9 depicts ELISA detection of PDGF Rβ phosphorylated at Y751. Human MG-63 osteosarcoma cells were incubated for 10 minutes in assay medium supplemented with 5% normal human serum and different concentrations of rhPDGF-BB ranging from 0.2 to 200 ng/mL. Cells were lysed and lysates analyzed with two sandwich ELISAs detecting phosphorylated PDGF Rβ and total PDGF Rβ. The concentration of phosphorylated receptor was normalized to the total concentration of receptor in the lysates.

FIGS. 10A and 10B depict ELISA detection of PDGF Rβ phosphorylated at Y751. Human MG-63 osteosarcoma cells were incubated for 10 minutes in assay medium supplemented with 5% normal human serum, 5 ng/mL rhPDGF-BB and different concentrations of anti-PDGF-BB antibodies ranging from 6.3 to 200 ng/mL. Cells were lysed and lysates analyzed with two sandwich ELISAs detecting phosphorylated PDGF Rβ. FIG. 10A depicts the results with goat anti-PDGF-BB antibody, while FIG. 10B depicts results with rabbit anti-PDGF-BB antibody.

FIGS. 11A and 11B depict ELISA detection of PDGF Rβ phosphorylated at Y751. Human MG-63 osteosarcoma cells were incubated for 10 minutes in assay medium supplemented with 5% normal human serum, 10 ng/mL rhPDGF-DD and different concentrations of anti-PDGF-BB antibodies ranging from 6.3 to 200 ng/mL. Cells were lysed and lysates analyzed with two sandwich ELISAs detecting phosphorylated PDGF Rβ. FIG. 10A depicts the results with goat anti-PDGF-BB antibody, while FIG. 10B depicts results with rabbit anti-PDGF-BB antibody.

### DETAILED DESCRIPTION

The present disclosure provides methods for detecting the presence of protein therapeutic neutralizing antibodies a serum sample, comprising: contacting a population of cells with: i) a serum sample that may contain the protein therapeutic neutralizing antibodies, and ii) the protein therapeutic, wherein the cells comprise a receptor for the protein therapeutic; and detecting a biomarker indicative of binding of the protein therapeutic with the receptor, and correlating the amount of the biomarker with the presence of protein therapeutic neutralizing antibodies.

### Methods for Detecting Neutralizing Antibodies

In other embodiments, the present disclosure provides methods for detecting the presence of growth factor neutralizing antibodies in a serum sample. For example, one embodiment of the present disclosure provides a method for detecting the presence of growth factor neutralizing antibodies in a serum sample, comprising contacting a population of cells with i) a serum sample that may contain the growth factor neutralizing antibodies, and growth factor, wherein the cells comprise a growth factor receptor; detecting an amount of a biomarker in the population of cells, wherein the biomarker indicates binding of the growth factor with the growth factor receptor, and correlating the amount of the biomarker with the presence of the growth factor neutralizing antibodies. According to the present methods, when growth factor neutralizing antibodies are present in the serum sample, the amount of the biomarker will be reduced compared to a serum sample that does not contain growth factor-neutralizing antibodies.

A serum sample of a subject may be suspected of containing growth factor neutralizing antibodies when the subject has received or is receiving a growth factor-containing therapeutic. For example, a growth factor can be administered to a subject by applying it directly to an area needing healing or regeneration. Generally, it is applied in a resorbable or non-resorbable carrier as a liquid or solid, and the site then covered with a bandage or nearby tissue. Growth factors that may administered to a subject include, without limitation, the growth factor is selected from the group consisting of PDGF, BMPs, EGF, fibroblast growth factor FGF, IGF, TGF-α, TGF-β, TNF-α, and VEGF.

The aforementioned growth factors can be obtained from human tissues or cells (e.g. platelets), produced by solid phase synthesis or produced by recombinant DNA technology. When obtained from natural sources, the growth factor can be obtained from a biological fluid. A biological fluid includes any treated or untreated fluid (including a suspension) associated with living organisms, particularly blood, including whole blood, warm or cold blood, and stored or fresh blood; treated blood, such as blood diluted with at least one physiological solution, including but not limited to saline, nutrient, and/or anticoagulant solutions; blood components, such as platelet concentrate (PC), platelet-rich plasma (PRP), platelet-poor plasma (PPP), platelet-free plasma, plasma, serum, fresh frozen plasma (FFP), components obtained from plasma, packed red cells (PRC), buffy coat (BC); blood products derived from blood or a blood component or derived from bone marrow; red cells separated from plasma and resuspended in physiological fluid; and platelets separated from plasma and resuspended in physiological fluid. The biological fluid may have been treated to remove some of the leukocytes before being processed. As used herein, blood product or biological fluid refers to the components described above, and to similar blood products or biological fluids obtained by other means and with similar properties.

The aforementioned growth factor receptor sites on the cells undergo phosphorylation upon binding to the growth factor. Thus, in some embodiments, the biomarker indicative of binding of the growth factor to the receptor is a phosphorylated growth factor receptor. While not being bound by any particular theory, it is believed that the level of phosphorylated growth factor in the cells will increase with increasing exposure to the growth factor, but the presence of growth factor-neutralizing antibodies in a serum sample will result in a decrease in the level of phosphorylated growth factor receptor. Advantageously, measurement of a phosphorylated growth factor receptor, as opposed to measuring downstream effects of growth factor signaling, is that this approach minimizes the effect of other bioactive molecules (for example growth factors and cytokines) that may be present in the serum sample.

Phosphorylated growth factor receptors can be detected by any technique known in the art, for example by Western blot analysis or by an ELISA assay. For a Western blot analysis, a sample of cells can be combined with the growth factor and incubated for a period of time. The cells are then lysed and the supernatant collected. Proteins are separated on a 5% Tris-HCl gel and transferred onto a PDVF membrane. An enzyme-linked immunosorbent (ELISA)-based assay provides a more quantitative method of detecting the presence of growth factor neutralizing antibodies. With an ELISA assay, the optical density of the sample may be measured and quantified.

In some embodiments, the step of contacting the cells with the growth factor and serum sample comprises incubating the cells in the serum sample. The cells may be incubated at a temperature of about 37° C. for a period of time sufficient to induce phosphorylation of the growth factor receptor. For example, the cells may be incubated for a period of time ranging from about 2 to about 30 minutes. In some embodiments, the cells are incubated for about 2 to about 10 minutes, about 5 to about 10 minutes, or for about 10 minutes

The population of cells can be any cells comprising at least one receptor for the particular growth factor being analyzed. In certain embodiments, the cells are human neonatal fibroblasts, while in other embodiments, the cells are MG-63 osteosarcoma cells.

In certain embodiments, when the cells are MG-63 osteosarcoma cells, the method comprises serum starving the cells prior to stimulating them with the growth factor during the contacting step. The cells may be serum starved for a period of time ranging from about 4 hours to about 48 hours, about 4 hours to about 24 hours, about 4 hours to about 16 hours, about 4 hours to about 12 hours, or about 6 hours to about 12 hours.

In certain embodiments, the serum sample is preincubated with the growth factor prior to the contacting step. While not being bound by theory, any growth factor neutralizing antibodies present in the sample will interact with and neutralize growth factor and neutralize it during the preincubation step. Thus, when the mixture of the preincubated serum and growth factor is incubated with the population of cells, neutralized growth factor will not induce phosphorylation of the receptor.

The concentration of the growth factor is effective for binding the growth factor receptor and thereby inducing formation of the biomarker, for example a concentration ranging from about 0.05 ng/mL to about 50 μg/mL. In other embodiments, the growth factor has a concentration ranging from about 0.5 ng/mL to about 100 ng/mL, about 1 ng/mL to about 50 ng/mL, about 1 ng/mL to about 20 ng/mL, or about 1 ng/mL to about 10 ng/mL. In other embodiments, the concentration of growth factor may be about 2.5 ng/mL, about 5 ng/mL, or about 10 ng/mL. In still other embodiments, the growth factor has a concentration ranging from about 0.01 μg/mL to about 50 μg/mL, For example, the concentration of growth factor may be in a range of about 0.1 μg/mL to about 50 μg/mL, or about 0.1 μg/mL to about 10 μg/mL. In other embodiments, the growth factor has a concentration of about 0.1 μg/mL to about 5 μg/mL, about 1 μg/mL to about 5 μg/mL, about 1 μg/mL, about 1.16 μg/mL, about 5 μg/mL or about 45 μg/mL. It is to be understood the aforementioned concentrations are merely examples of particular embodiments, and that the concentration of growth factor may be within any of the concentration ranges recited above.

The cells may be contacted with PDGF and the sample while in a suspension or while adhered to cell culture plates. In some embodiments, a coating for the culture plates is used, such as poly-L-lysine. When the cells are adhered to culture plates during the contacting step, the cell density may be in, in some embodiments, in a range of about 1×10⁴ to about 1×10⁵ cells/cm². In other embodiments, the cell density is in a range of about 1×10⁴ to about 5×10⁴ cells/cm², about 1×10⁴ to about 4×10⁴ cells/cm², about 2×10⁴ to about 4×10⁴ cells/cm², or about 2×10⁴ cells/cm². In some embodiments, the cells are lysed after the contacting step. The cells may be lysed while still adhered to the culture plates.

In certain embodiments, the method includes a positive control, a negative control, or both. Accordingly, a negative control can comprise negative base pool (NBP; serum pooled from donors not receiving the growth factor therapeutic), and a positive control can comprise a growth factor neutralizing antibody. Accordingly, the method can include comparative analysis of the serum sample against positive and negative controls in order to assess the presence of PDGF neutralizing antibodies.

More particularly, the methods may further comprise determining a floating cut point for detecting the presence of neutralizing antibodies. A floating cut point is useful in the event that inter assay and inter-analyst variations exist. In some embodiments, the floating cut point is determined based on a negative base pool, and the floating cut point is correlated with the presence of growth factor neutralizing antibodies. Thus, the amount of the biomarker in the population of cells treated with the test serum sample can be compared to the floating cut point.

The floating cut point in particular embodiments is determined by contacting a second population of cells with i) a negative base pool sample, and ii) the growth factor, wherein the cells comprise the growth factor receptor, and detecting an amount of the biomarker in the second population of cells. In particular embodiments, the floating cut point is tied to a statistical measure of the negative base pool. For example, the statistical measure can be a standard deviation, a standard error, a mean, a median, a median absolute deviation, a fit parameter, or the like. Further, in some embodiments, a multiplicative factor may be assigned in calculating the cut point.

The detected amount of the biomarker, such as phosphorylated growth factor receptor, in the serum sample can be evaluated compared to the floating cut point. For example, when a detected amount of the biomarker in the sample is greater than about 80% of the floating cut point, then the serum sample does not contain appreciable quantities of the growth factor neutralizing antibodies. In other embodiments, when the detected amount of the biomarker in the serum sample is greater than about 85%, about 90% about 95%, about 96%, about 97%, about 98%, about 99% or about 100% of the floating cut point, then the serum sample does not contain appreciable quantities of the growth factor neutralizing antibodies.

In yet another embodiment, the disclosure provides a method of determining the presence of growth factor neutralizing antibodies in a subject who has received a treatment comprising PDGF, comprising: providing a serum sample from the subject, contacting a population of cells with i) a serum sample, and ii) the growth factor, wherein the cells comprise a growth factor receptor; and detecting an amount of a biomarker in the population of cells, wherein the biomarker is indicative of binding of the growth factor to the growth factor receptor, and correlating the amount of the biomarker with the presence of the growth factor neutralizing antibodies. When the serum sample from the subject is determined to contain anti-growth factor antibodies, then the method further comprises the step of discontinuing treatment with the growth factor. When the serum sample does not contain neutralizing growth factor antibodies, the method further comprises continuing the treatment of the subject with the growth factor.

### Methods for Detecting PDGF-Neutralizing Antibodies

More particularly, the present disclosure provides methods for detecting the presence of PDGF neutralizing antibodies in a serum sample. For example, one embodiment of the present disclosure provides a method for detecting the presence of PDGF neutralizing antibodies in a serum sample, comprising contacting a population of cells with i) a serum sample that may contain the PDGF neutralizing antibodies, and ii) PDGF, wherein the cells comprise a PDGF receptor; and detecting an amount of a biomarker in the population of cells, wherein the biomarker indicates binding of the PDGF with the PDGF receptor, and correlating the amount of the biomarker with the presence of the PDGF neutralizing antibodies. According to the present methods, when PDGF-neutralizing antibodies are present in the serum sample, the amount of the biomarker will be reduced compared to a serum sample that does not contain PDGF-neutralizing antibodies.

A serum sample of a subject may be suspected of containing PDGF-neutralizing antibodies when the subject has received or is receiving a PDGF-containing therapeutic. For example, PDGF can be administered to a subject by applying it directly to an area needing healing or regeneration. Generally, it is applied in a resorbable or non-resorbable carrier as a liquid or solid, and the site then covered with a bandage or nearby tissue. An amount of PDGF sufficient to promote bone growth or tissue healing is generally a concentration of about 0.1 to about 1.0 mg/mL of PDGF. In certain embodiments, the concentration of PDGF is about 0.3 mg/mL.

PDGF can be obtained from human tissues or cells (e.g. platelets), produced by solid phase synthesis or produced by recombinant DNA technology. When obtained from natural sources, the PDGF can be obtained from a biological fluid. A biological fluid includes any treated or untreated fluid (including a suspension) associated with living organisms, particularly blood, including whole blood, warm or cold blood, and stored or fresh blood; treated blood, such as blood diluted with at least one physiological solution, including but not limited to saline, nutrient, and/or anticoagulant solutions; blood components, such as platelet concentrate (PC), platelet-rich plasma (PRP), platelet-poor plasma (PPP), platelet-free plasma, plasma, serum, fresh frozen plasma (FFP), components obtained from plasma, packed red cells (PRC), buffy coat (BC); blood products derived from blood or a blood component or derived from bone marrow; red cells separated from plasma and resuspended in physiological fluid; and platelets separated from plasma and resuspended in physiological fluid. The biological fluid may have been treated to remove some of the leukocytes before being processed. As used herein, blood product or biological fluid refers to the components described above, and to similar blood products or biological fluids obtained by other means and with similar properties. In an embodiment, the PDGF is obtained from platelet-rich plasma (PRP). The preparation of PRP is described in, e.g., U.S. Pat. Nos. 6,649,072, 6,641,552, 6,613,566, 6,592,507, 6,558,307, 6,398,972, and 5,599,558, which are incorporated herein by reference.

When produced by recombinant technology, the recombinant factor can be a recombinant heterodimer, made by inserting into cultured prokaryotic or eukaryotic cells DNA sequences encoding both subunits of the factor, and then allowing the translated subunits to be processed by the cells to form a heterodimer (e.g., PDGF-AB). Alternatively, DNA encoding just one of the subunits (e.g., PDGF B-chain or A-chain) can be inserted into cells, which then are cultured to produce the homodimeric factor (e.g., PDGF-BB or PDGF-AA homodimers). PDGF for use in the methods of the invention includes PDGF homo- and heterodimers, for example, PDGF-AA, PDGF-BB, PDGF-AB, PDGF-CC, and PDGF-DD, and combinations and derivatives thereof. In some embodiments, the PDGF is PDGF-BB.

In some embodiments, the PDGF is rh PDGF, which can be prepared using the following procedures. Platelet-derived growth factor (PDGF) derived from human platelets contains two polypeptide sequences (PDGF-B and PDGF-A polypeptides; Antoniades, H. N. and Hunkapiller, M., Science 220:963-965, 1983). PDGF-B is encoded by a gene localized on chromosome 7 (Betsholtz, C. et al., Nature 320:695-699), and PDGF-A is encoded by the sis oncogene (Doolittle, R. et al., Science 221:275-277, 1983) localized on chromosome 22 (Dalla-Favera, R., Science 218:686-688, 1982). The sis gene encodes the transforming protein of the Simian Sarcoma Virus (SSV) which is closely related to PDGF-2 polypeptide. The human cellular c-sis also encodes the PDGF-A chain (Rao, C. D. et al., Proc. Natl. Acad. Sci. USA 83:2392-2396, 1986). Because the two polypeptide chains of PDGF are coded by two different genes localized in separate chromosomes, the possibility exists that human PDGF consists of a disulfide-linked heterodimer of PDGF-B and PDGF-A, or a mixture of the two homodimers (PDGF-BB homodimer and PDGF-AA homodimer), or a mixture of the heterodimer and the two homodimers.

Mammalian cells in culture infected with the Simian Sarcoma Virus, which contains the gene encoding the PDGF-A chain, were shown to synthesize the PDGF-A polypeptide and to process it into a disulfide-linked homodimer (Robbins et al., Nature 305:605-608, 1983). In addition, the PDGF-A homodimer reacts with antisera raised against human PDGF. Furthermore, the functional properties of the secreted PDGF-A homodimer are similar to those of platelet-derived PDGF in that it stimulates DNA synthesis in cultured fibroblasts, it induces phosphorylation at the tyrosine residue of a 185 kD cell membrane protein, and it is capable of competing with human (sup.125I)-PDGF for binding to specific cell surface PDGF receptors (Owen, A. et al., Science 225:54-56, 1984). Similar properties were shown for the sis/PDGF-A gene product derived from cultured normal human cells (for example, human arterial endothelial cells), or from human malignant cells expressing the sis/PDGF-2 gene (Antoniades, H. et al., Cancer Cells 3:145-151, 1985).

The recombinant PDGF-B homodimer is obtained by the introduction of cDNA clones of α-sis/PDGF-B gene into mouse cells using an expression vector. The c-sis/PDGF-B clone used for the expression was obtained from normal human cultured endothelial cells (Collins, T., et al., Nature 216:748-750, 1985). In certain embodiments, the PDGF used in any of the present methods is rhPDGF-BB.

There are two structurally related PDGF receptors: PDGF Rα and PDGF Rβ. The receptors are independently regulated, but have been found to be expressed together on fibroblasts, smooth muscle cells and neurons. Other cell types, such as platelets and rat liver endothelial cells express only PDGF Rα, while mouse capillary endothelial cells express only PDGF Rβ. The receptors have roughly equivalent binding for PDGF-BB. Binding of PDGF-BB induces the formation of homodimers and/or heterodimers of the receptors (Heldin and Westermark). Depending on the number and ratio of the receptors present on a cell, the cell will be more or less responsive to the different PDGF family members. rhPDGF-BB has the ability to bind with high affinity to both receptors, providing it with unique properties within the PDGF family. Depending on the location of an injury, different cell types that respond to rhPDGF-BB are stimulated. Accordingly, in some embodiments, the PDGF receptor is PDGF Rα, PDGF Rβ, or a combination thereof.

PDGF receptors are members of the receptor-tyrosine kinase family and upon ligand-induced dimerization have intrinsic kinase activity, which results in autophosphorylation. The phosphorylated receptors act as a docking site for kinases, phosphatases, and adaptor molecules. The tyrosine at position 751 (Y751) of human PDGF Rβ has been shown to be a docking site for phosphinositide 3-kinase (Kazluskas and Cooper, 1990), which is involved in PDGF-BB induced cell proliferation and migration (Bornfelt et al., 1995). The phosphorylation of the PDGF receptors is followed by a cascade of intracellular signal transduction that ultimately results in cell activities such as mitosis or migration. Because phosphorylation of the PDGF receptors is a highly specific activity of PDGF, the level of phosphorylation is expected to able a reliable outcome measure for the presence of PDGF-neutralizing antibodies in serum samples.

Accordingly, in certain embodiments, the biomarker indicative of binding of the PDGF to the PDGF receptor is a phosphorylated PDGF receptor, for example, phosphorylated PDGF Rβ. While not being bound by any particular theory, it is believed that the level of phosphorylated PDGF Rβ in the cells will increase with rhPDGF-BB exposure, but the presence of PDGF-neutralizing antibodies in a serum sample will result in a decrease in the level of phosphorylated PDGF Rβ. Advantageously, measurement of phosphorylated PDGF Rβ, as opposed to measuring downstream effects of PDGF signaling, such as cell proliferation or migration, is that this approach minimizes the effect of other bioactive molecules (for example growth factors and cytokines) that may be present in the serum sample. Thus, the only molecules expected to affect the level of phosphorylated PDGF Rβ (Y751) are members of the PDGF family of growth factors.

Phosphorylated PDGF receptors can be detected by any technique known in the art, for example by Western blot analysis or by an ELISA assay. For a Western blot analysis, a sample of cells can be combined with PDGF and incubated for a period of time. The cells are then lysed and the supernatant collected. Proteins are separated on a 5% Tris-HCl gel and transferred onto a PDVF membrane. Phosphorylated PDGF Rβ can be detected using rabbit anti-phospho-PDGF Rβ (Y751) antibody, and HRP-conjugated goat anti-rabbit IgG antibody can be used as a secondary reagent.

An ELISA-based assay provides a more quantitative method of detecting the presence of PDGF neutralizing antibodies. For example, a commercially available kit, DUOSet® IC kit from R&D Systems (Catalog No. DYC3096-2), can detect phosphorylated PDGF Rβ (Y751). The ELISA plates are coated with a capture antibody (e.g. goat anti-human PDGF Rβ) and blocked with a BSA solution. The manufacturer's standards and cell lysates are then added and incubated. After washing unbound material from the plates, a biotinylated detected antibody capable of recognizing PDGF Rβ phosphorylated at Y751 is used to detect the presence of the phosphorylated receptor.

In some embodiments, the step of contacting the cells with the PDGF and serum sample comprises incubating the cells in the serum sample. The cells may be incubated at a temperature of about 37° C. for a period of time sufficient to induce phosphorylation of the PDGF receptor. For example, the cells may be incubated for a period of time ranging from about 2 to about 30 minutes. In some embodiments, the cells are incubated for about 2 to about 10 minutes, about 5 to about 10 minutes, or for about 10 minutes

The population of cells can be any cells comprising at least one PDGF receptor. In some embodiments, the PDGF receptor is PDGF Rα, PDGF Rβ, or a combination thereof. In other embodiments, the PDGF receptor is PDGF R. In certain embodiments, the cells are human neonatal fibroblasts or MG-63 osteosarcoma cells.

In certain embodiments, when the cells are MG-63 osteosarcoma cells, the method comprises serum starving the cells prior to stimulating them with the PDGF during the contacting step. The cells may be serum starved for a period of time ranging from about 4 hours to about 48 hours, about 4 hours to about 24 hours, about 4 hours to about 16 hours, about 4 hours to about 12 hours, or about 6 hours to about 12 hours.

In certain embodiments, the serum sample is preincubated with the PDGF prior to the contacting step. While not being bound by theory, any PDGF neutralizing antibodies present in the sample will interact with and neutralize PDGF and neutralize it during the preincubation step. Thus, when the mixture of the preincubated serum and PDGF is incubated with the population of cells, neutralized PDGF will not induce phosphorylation of the receptor.

The concentration of PDGF is effective for binding the PDGF receptor and thereby inducing formation of the biomarker, for example a concentration ranging from about 0.5 ng/mL to about 50 μg/mL. In other embodiments, the PDGF has a concentration ranging from about 0.5 ng/mL to about 100 ng/mL, about 1 ng/mL to about 50 ng/mL, about 1 ng/mL to about 20 ng/mL, or about 1 ng/mL to about 10 ng/mL. In other embodiments, the concentration of PDGF may be about 2.5 ng/mL, about 5 ng/mL, or about 10 ng/mL. In still other embodiments, the PDGF has a concentration ranging from about 0.01 μg/mL to about 50 μg/mL. For example, the concentration of PDGF may be in a range of about 0.1 μg/mL to about 50 μg/mL, or about 0.1 μg/mL to about 10 μg/mL. In other embodiments, the PDGF has a concentration of about 0.1 μg/mL to about 5 μg/mL, about 1 μg/mL to about 5 μg/mL, about 1 μg/mL, about 1.16 μg/mL, about 5 μg/mL or about 45 μg/mL. It is to be understood the aforementioned concentrations are merely examples of particular embodiments, and that the concentration of PDGF may be within any of the concentration ranges recited above.

The cells may be contacted with PDGF and the sample while in a suspension or while adhered to cell culture plates. In some embodiments, a coating for the culture plates is used, such as poly-L-lysine. When the cells are adhered to culture plates during the contacting step, the cell density may be in, in some embodiments, in a range of about 1×10⁴ to about 1×10⁵ cells/cm². In other embodiments, the cell density is in a range of about 1×10⁴ to about 5×10⁴ cells/cm², about 1×10⁴ to about 4×10⁴ cells/cm², about 2×10⁴ to about 4×10⁴ cells/cm², or about 2×10⁴ cells/cm². In some embodiments, the cells are lysed after the contacting step. The cells may be lysed while still adhered to the culture plates.

In certain embodiments, the method includes a positive control, a negative control, or both. For example, goat-anti-PDGF-BB antibody and rabbit anti-PDGF-BB antibody are capable of neutralizing PDGF-BB. Accordingly, a negative control can comprise negative base pool (NBP; serum pooled from donors not receiving a PDGF therapeutic), a positive control can comprise a PDGF-BB antibody, and control of NBP without PDGF. Accordingly, the method can include comparative analysis of the serum sample against positive and negative controls in order to assess the presence of PDGF neutralizing antibodies.

More particularly, the methods may further comprise determining a floating cut point for detecting the presence of PDGF neutralizing antibodies. A floating cut point is useful in the event that inter-assay and inter-analyst variations exist. In some embodiments, the floating cut point is determined based on a negative base pool, and the floating cut point is correlated with the presence of PDGF neutralizing antibodies. Thus, the amount of the biomarker, such as phosphorylated PDGF receptor, in the population of cells treated with the test serum sample can be compared to the floating cut point.

The floating cut point in particular embodiments is determined by contacting a second population of cells with i) a negative base pool sample, and ii) PDGF, wherein the cells comprise a PDGF receptor, and detecting an amount of the biomarker in the second population of cells. In particular embodiments, the floating cut point is tied to a statistical measure of the negative base pool. For example, the statistical measure can be a standard deviation, a standard error, a mean, a median, a median absolute deviation, a fit parameter, or the like. Further, in some embodiments, a multiplicative factor may be assigned in calculating the cut point.

The detected amount of the biomarker, such as phosphorylated PDGF receptor, in the serum sample can be evaluated compared to the floating cut point. For example, when a detected amount of the biomarker in the sample is greater than about 80% of the floating cut point, then the serum sample does not contain appreciable quantities of PDGF neutralizing antibodies. In other embodiments, when the detected amount of the biomarker in the serum sample is greater than about 85%, about 90% about 95%, about 96%, about 97%, about 98%, about 99% or about 100% of the floating cut point, then the serum sample does not contain appreciable quantities of PDGF neutralizing antibodies.

In yet another embodiment, the disclosure provides a method of determining the presence of PDGF neutralizing antibodies in a subject who has received a treatment comprising PDGF, comprising: providing a serum sample from the subject, contacting a population of cells with i) a serum sample, and ii) the PDGF, wherein the cells comprise a PDGF receptor; and detecting an amount of a biomarker in the population of cells, wherein the biomarker is indicative of binding of the PDGF to the PDGF receptor, and correlating the amount of the biomarker with the presence of the PDGF neutralizing antibodies. When the serum sample from the subject is determined to contain anti-PDGF antibodies, then the method further comprises the step of discontinuing treatment with the PDGF. When the serum sample does not contain neutralizing growth factor antibodies, the method further comprises continuing the treatment of the subject with PDGF.

### Examples

Kinetics and dose dependence of PDGF Rβ phosphorylation Western Blot Assay

The kinetics and dose dependence of PDGF Rβ phosphorylation in human neonatal dermal fibroblasts was analyzed. Human neonatal dermal fibroblasts were grown to 85-95% confluence then serum starved for 2-4 hours. After serum starvation, the cells were trypsinized, counted, washed, and resuspended at a density of 2×10⁷ cells/mL in DPBS.

For the phosphorylation kinetics studies, the cells were distributed in microtubes at 1×10⁶ cells/tube. With the exception of an unstimulated (control) sample, rhPDGF-BB was added to a final concentration of 45 μg/mL. The cells were incubated in a 37° C. water bath for 2, 5, 10, or 30 minutes and then lysed with RIPA buffer containing protease and phosphatase inhibitors. The lysates were sonicated and centrifuged at 1,500×g. The supernatants were collected and stored at −80° C. until Western Blot analyses were performed.

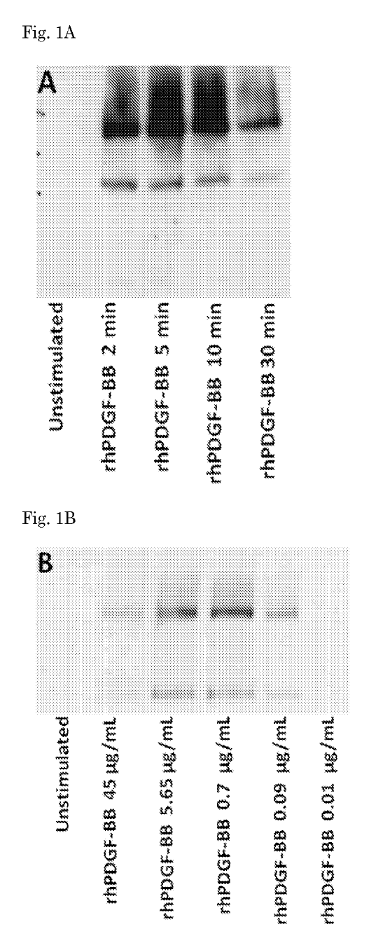

For the dose dependence studies, the cells were distributed in microtubes at 1×10⁶ cells/tube and rhPDGF-BB was added to final concentrations of 0, 45, 5.65, 0.7, 0.09, and 0.1 μg/mL. The cells were incubated in a 37° C. water bath for 2 minutes and then lysed with RIPA buffer containing protease and phosphatase inhibitors. The lysates were sonicated and centrifuged at 1,500×g. The supernatant were collected and stored at −80° C. until Western Blot analyses were performed. Proteins were separated on a 5% Tris-HCl gel and transferred onto a PVDF membrane. For Western blotting, rabbit anti-phospho-PDGF Rβ (Y751) antibody (R&D Systems Catalog # AF1767) was used at 0.5 mg/mL. The secondary reagent, HRP-conjugated goat anti-rabbit IgG antibody (KPL Catalog #074-1506), was used at 1:20,000 dilution. Membranes were developed using ECL (Pierce/Thermo Scientific Catalog #32106). As shown in FIGS. 1A and 1B, rhPDGF-BB induced PDGF Rβ phosphorylation in human neonatal dermal fibroblast in a time and dose dependent manner. Maximal phosphorylation of PDGF Rβ occurs between about 2-10 minutes at a concentration of approximately 1 μg/mL.

Based on these preliminary Western blotting data, additional human neonatal dermal fibroblast lysates were produced using cells stimulated with rhPDGF-BB at a concentration of 1.16 μg/mL for 2 minutes. Cells were also stimulated with negative base pool (NBP) and NBP spiked with rhPDGF-BB at 1.16 μg/mL. The level of phosphorylated PDGF Rβ (Y751) in each lysate was determined by Western blotting. As shown in FIG. 2, Western analysis did not detect phosphorylation of PDGF Rβ at Y751 in human neonatal dermal fibroblasts incubated in the presence of NBP at 1:20 (5%), 1:50 (2%), or 1:100 (1%) dilution. On the other hand, phosphorylation of the receptor was detected when rhPDGF-BB was present.

### ELISA-Based Assay

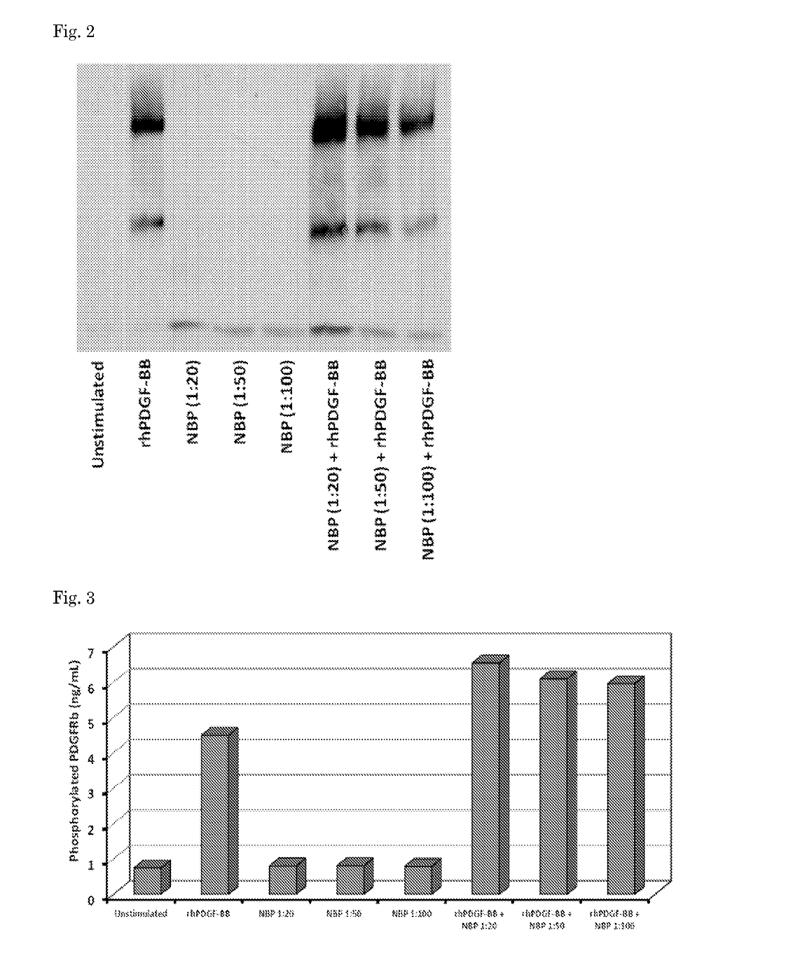

As described above, the initial assay development focused on monitoring the phosphorylation of the receptor was made by semi-quantitative Western blotting using cell lysates from neonatal dermal fibroblasts. A more quantitative approach was attempted by adapting a DuoSet® IC kit from R&D systems (Catalog # DYC3096-2) that detects phosphorylated PDGF Rβ (Y751). Lysates prepared in NBP spiked with rhPDGF-BB at 1.16 μg/mL as described above were analyzed using the DuoSet kit following the manufacturer's recommended instructions. Briefly, ELISA plates were coated with goat anti-human PDGF Rβ antibody (capture antibody) then blocked with 1% BSA solution. The manufacturer's standards and cell lysates were added and incubated for 2 hours. After washing unbound material, a biotinylated detection antibody recognizing PDGF Rβ phosphorylated at Y751 was used to detect phosphorylated protein utilizing a standard streptavidin-HRP format. The concentration of phosphorylated PDGF Rβ in each lysate was calculated using a four-parameter logistic regression curve fitting with the standards. The results obtained with the DuoSet® IC kit (FIG. 3) were similar to those obtained by Western blotting. MG-63 Osteosarcoma cell lysates

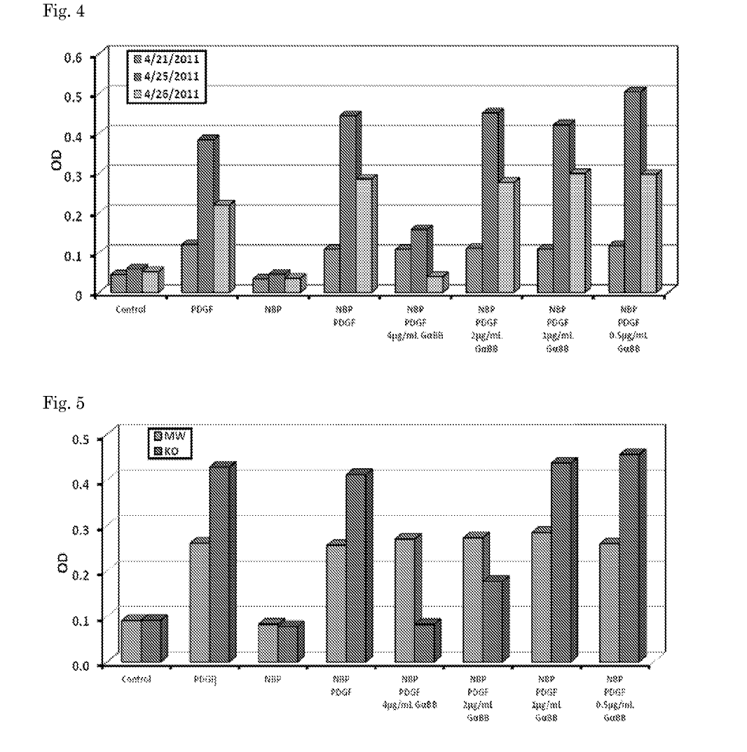

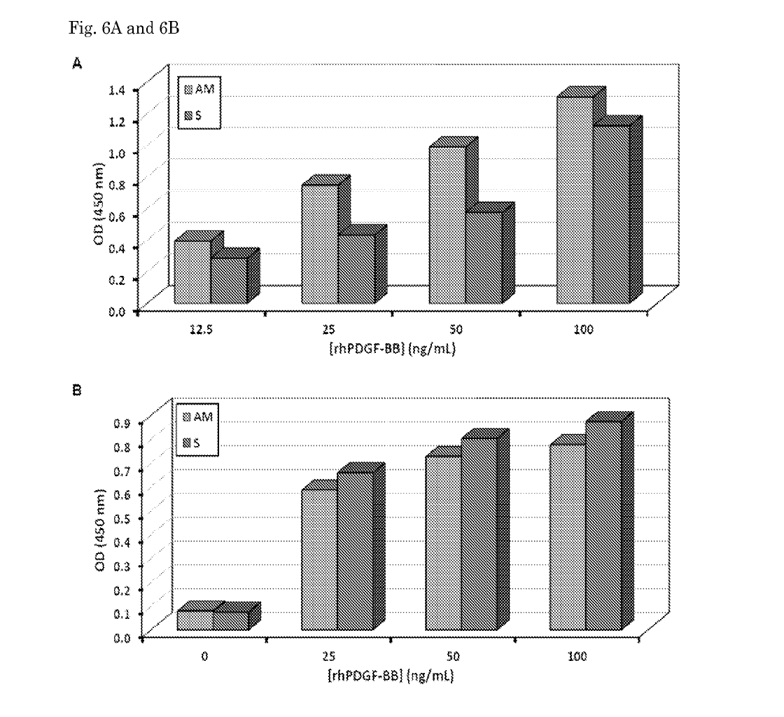

The above studies are based on detection of the phosphorylated receptor was performed using human neonatal dermal fibroblasts. However, these cells are not an established cell line and cells from different donors may exhibit different levels of receptor expression and responsiveness to rhPDGF-BB stimulation. Accordingly, we use the MG-63 osteosarcoma cell line to study the quantification of phosphorylated receptor using the DuoSet® ELISA. Cell lysates were prepared following the same protocol developed for the human neonatal fibroblasts. Preparation of cell lysates following these methods has proven to be highly variable and unreliable with significant inter-assay (FIG. 4) and inter-analyst (FIG. 5) differences.

In the process of development of the ELISA-based assay, it was determined that trypsinization of the cells immediately prior to stimulation with rhPDGF-BB was suboptimal. In an attempt to optimize cellular response and performance of the assay, the protocol was modified to include stimulation of the MG-63 cells while still attached to the cell culture surface followed by lysis of the cells still on the cell culture plates. This modified protocol produced more consistent results indicating a dose dependent increase of phosphorylation of the receptor (FIGS. 6A and 6B).

Further development of the assay conditions assessed the impact of variables such as concentration of rhPDGF-BB for cell stimulation, duration of the cell stimulation, cell density, and pre-treatment of the cell culture surfaces. Another goal of assay development was to optimize the assay to allow a reasonable throughput by scaling down the assay from 56 cm² cell culture dishes to 6-well and 12-well plates.

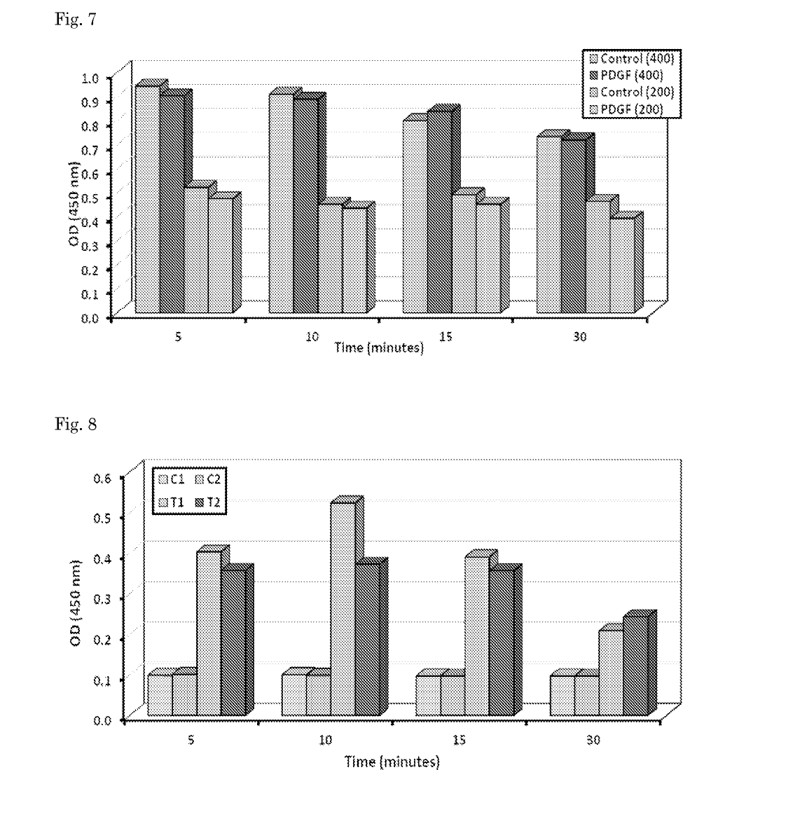

When the MG-63 cells were serum-starved overnight prior to stimulation with rhPDGF-BB, some of the cells detached from the culture dishes resulting in inconsistent results. Cell detachment was prevented by pre-coating the tissue culture surfaces with poly-L-lysine prior to cell seeding. Additionally, even in poly-L-lysine-coated dishes, when cells were seeded at high density (4×10⁴ cells/cm²), stimulation with rhPDGF-BB after overnight serum starvation, resulted in morphological changes and detachment of the cells; these changes were not observed or were less noticeable when the cells were seeded at 2×10⁴ cells/cm² (FIG. 7).

Finally, even when seeded at lower density (2×10⁴ cells/cm²) exposure of the cells to rhPDGF-BB for longer than 10 minutes resulted in morphologic changes, and partial detachment of the cells yielding lower signal-to-noise ratios for the assay (FIG. 8). A stimulation time of 10 minutes was chosen as optimal to maximize the signal-to-noise ratio.

Determination of the Effective Dose of rhPDGF-BB and Linearity of the Assay

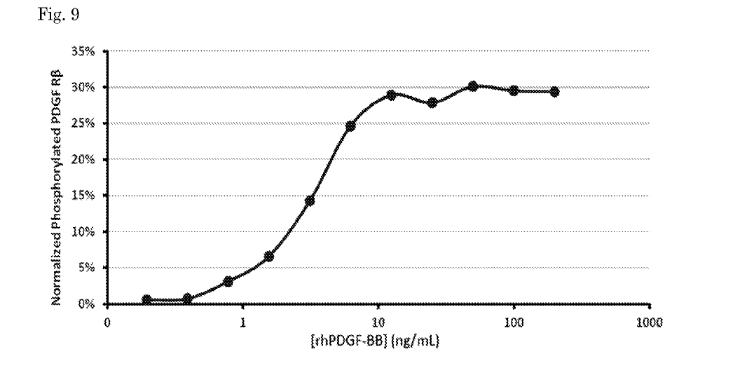

The dose-dependence of the receptor phosphorylation was assessed in duplicate using two different lots of human serum. As shown in FIG. 9, phosphorylation is dose-dependent, reaches saturation at a concentration of 10 ng/mL and the linear range of the assay is between 1 and 10 ng/mL. Four-parameter logistic curve fit determined that the EC50 is approximately 2.5 ng/mL.

### Inhibition of the Phosphorylation by Neutralizing Antibodies

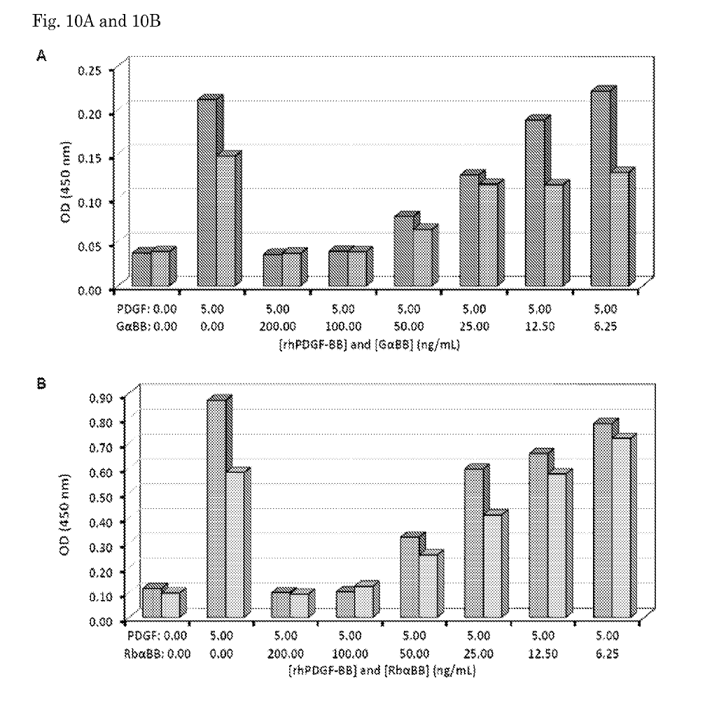

The dose-dependence of the inhibition of receptor phosphorylation by anti-PDGF-BB neutralizing antibodies was assessed in duplicate using two different lots of human serum and two different neutralizing antibodies. The antibodies used were an affinity-purified goat anti-PDGF-BB polyclonal antibody (GαBB; R&D Systems) and an affinity-purified rabbit anti-PDGF-BB polyclonal antibody (RαBB; LifeSpan Biosciences). As shown in FIGS. 10A and 10B, inhibition of rhPDGF-BB-induced receptor phosphorylation is dose-dependent for both antibodies. When the concentration of rhPDGF-BB used to stimulate the cells is 5.00 ng/mL saturation is reached at approximately 100 ng/mL.

### Specificity of the Inhibition of Phosphorylation by Neutralizing Antibodies

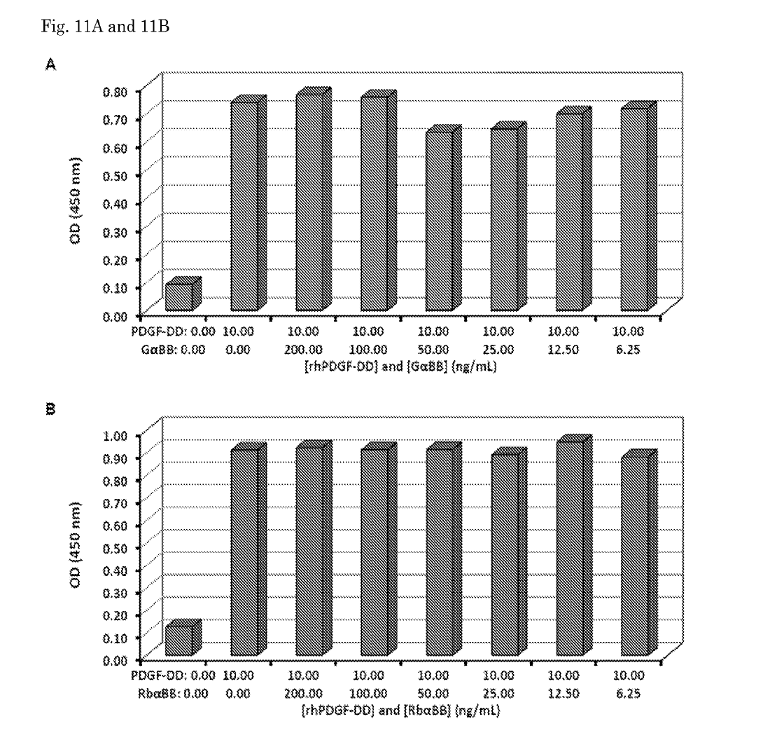

The specificity of the inhibition of phosphorylation of the receptor by anti-PDGF-BB neutralizing antibodies was assessed in duplicate using two different lots of human serum and the same neutralizing antibodies described above, but in these experiments the cells were stimulated with rhPDGF-DD which also triggers phosphorylation of the PDGF receptor β. As shown in FIGS. 11A and 11B, anti-PDGF-BB antibodies are specific and do not inhibit rhPDGF-DD-induced receptor phosphorylation.

### Determination of the Assay Cut Point

The levels of rhPDGF-BB-induced receptor phosphorylation in MG-63 cell lysates were measured after treatment with rhPDGF-BB preincubated with 30 baseline serum samples from patients receiving a PDGF-containing therapeutic. Each serum sample was tested twice (in two separate days) by two analysts (A and B) for a total of four assay runs. Each assay run included 5 controls: negative base pool (NBP; pooled serum from 10 normal subjects), high positive control (HPC; 1,000 ng/mL GαBB in NBP), medium positive control (MPC; 350 ng/mL GαBB in NBP), low positive control (LPC; 125 ng/mL GαBB in NBP), and unstimulated cells (US; incubated in NBP but in the absence of rhPDGF-BB). Table 1 shows the average OD (450 nm) for triplicate readings of duplicate wells from each control and sample in the Phospho-PDGF Rβ ELISA. Data underlined were considered outliers for the dataset using the box-plot approach that identifies points above the 75th percentile plus 1.5 times the interquartile range (high outliers) and below the 25th percentile minus 1.5 times the interquartile range (low outliers) and not included in the analysis for the cut point calculations.

TABLE 1

| | A Day 1 | | | A Day 2 | | | B Day 1 | | | B day 2 | | |
|---|---|---|---|---|---|---|---|---|---|---|---|---|
| | Mean | SD | CV | Mean | SD | CV | Mean | SD | CV | Mean | SD | CV |
| NBP | 0.1084 | 0.0227 | 21% | 0.1658 | 0.0314 | 19% | 0.1593 | 0.0304 | 19% | 0.1033 | 0.0161 | 16% |
| LPC | 0.0799 | 0.0009 | 1% | 0.1017 | 0.0034 | 3% | 0.1306 | 0.0006 | 0% | 0.0667 | 0.0015 | 2% |
| MPC | 0.0703 | 0.0010 | 1% | 0.0662 | 0.0013 | 2% | 0.0603 | 0.0017 | 3% | 0.0441 | 0.0010 | 2% |
| HPC | 0.0314 | 0.0013 | 4% | 0.0316 | 0.0024 | 7% | 0.0455 | 0.0027 | 6% | 0.0362 | 0.0014 | 4% |
| US | 0.0228 | 0.0013 | 6% | 0.0276 | 0.0006 | 2% | 0.0414 | 0.0040 | 10% | 0.0345 | 0.0002 | 1% |
| 1 | 0.1278 | 0.0037 | 3% | 0.2196 | 0.0037 | 2% | 0.1377 | 0.0027 | 2% | 0.1005 | 0.0018 | 2% |
| 2 | 0.1422 | 0.0021 | 1% | 0.2589 | 0.0088 | 3% | 0.1915 | 0.0011 | 1% | 0.1475 | 0.0056 | 4% |
| 3 | 0.1404 | 0.0055 | 4% | 0.2522 | 0.0056 | 2% | 0.1638 | 0.0037 | 2% | 0.1171 | 0.0041 | 3% |
| 4 | 0.1439 | 0.0044 | 3% | 0.2618 | 0.0028 | 1% | 0.2057 | 0.0026 | 1% | 0.1362 | 0.0033 | 2% |
| 5 | 0.1518 | 0.0093 | 6% | 0.2534 | 0.0039 | 2% | 0.2145 | 0.0028 | 1% | 0.1219 | 0.0041 | 3% |
| 6 | 0.1416 | 0.0016 | 1% | 0.2439 | 0.0045 | 2% | 0.2135 | 0.0005 | 0% | 0.1331 | 0.0009 | 1% |
| 7 | 0.1479 | 0.0023 | 2% | 0.2459 | 0.0069 | 3% | 0.1871 | 0.0028 | 1% | 0.1297 | 0.0016 | 1% |
| 8 | 0.1430 | 0.0010 | 1% | 0.2663 | 0.0061 | 2% | 0.1771 | 0.0014 | 1% | 0.1237 | 0.0012 | 1% |
| 9 | 0.1499 | 0.0031 | 2% | 0.2267 | 0.0082 | 4% | 0.1896 | 0.0004 | 0% | 0.1123 | 0.0040 | 4% |
| 10 | 0.1544 | 0.0039 | 2% | 0.2618 | 0.0033 | 1% | 0.1813 | 0.0041 | 2% | 0.1146 | 0.0009 | 1% |
| 11 | 0.1202 | 0.0037 | 3% | 0.2107 | 0.0049 | 2% | 0.2963 | 0.0004 | 0% | 0.1438 | 0.0018 | 1% |
| 12 | 0.1207 | 0.0013 | 1% | 0.1924 | 0.0020 | 1% | 0.1600 | 0.0043 | 3% | 0.1069 | 0.0017 | 2% |
| 13 | 0.1158 | 0.0032 | 3% | 0.2271 | 0.0024 | 1% | 0.3002 | 0.0014 | 0% | 0.1275 | 0.0021 | 2% |
| 14 | 0.1141 | 0.0033 | 3% | 0.2154 | 0.0052 | 2% | 0.1999 | 0.0011 | 1% | 0.1180 | 0.0052 | 4% |
| 15 | 0.1213 | 0.0032 | 3% | 0.2364 | 0.0028 | 1% | 0.2453 | 0.0019 | 1% | 0.1460 | 0.0036 | 2% |
| 16 | 0.1407 | 0.0045 | 3% | 0.2066 | 0.0007 | 0% | 0.1981 | 0.0056 | 3% | 0.1067 | 0.0030 | 3% |
| 17 | 0.1455 | 0.0020 | 1% | 0.2141 | 0.0014 | 1% | 0.2243 | 0.0029 | 1% | 0.1105 | 0.0037 | 3% |
| 18 | 0.1537 | 0.0019 | 1% | 0.1967 | 0.0019 | 1% | 0.1376 | 0.0021 | 2% | 0.1256 | 0.0031 | 2% |
| 19 | 0.1491 | 0.0019 | 1% | 0.2353 | 0.0030 | 1% | 0.2335 | 0.0015 | 1% | 0.1054 | 0.0028 | 3% |
| 20 | 0.1547 | 0.0019 | 1% | 0.2037 | 0.0035 | 2% | 0.1468 | 0.0021 | 1% | 0.1602 | 0.0019 | 1% |
| 21 | 0.1354 | 0.0029 | 2% | 0.1830 | 0.0042 | 2% | 0.2332 | 0.0042 | 2% | 0.1235 | 0.0016 | 1% |
| 22 | 0.1294 | 0.0037 | 3% | 0.1671 | 0.0027 | 2% | 0.1962 | 0.0042 | 2% | 0.0994 | 0.0028 | 3% |
| 23 | 0.1273 | 0.0016 | 1% | 0.1788 | 0.0057 | 3% | 0.2156 | 0.0051 | 2% | 0.1142 | 0.0020 | 2% |
| 24 | 0.1206 | 0.0009 | 1% | 0.1845 | 0.0008 | 0% | 0.2118 | 0.0032 | 1% | 0.1084 | 0.0014 | 1% |
| 25 | 0.1165 | 0.0006 | 0% | 0.1769 | 0.0025 | 1% | 0.1597 | 0.0027 | 2% | 0.1070 | 0.0046 | 4% |
| 26 | 0.1074 | 0.0008 | 1% | 0.2036 | 0.0031 | 2% | 0.1537 | 0.0027 | 2% | 0.1139 | 0.0025 | 2% |
| 27 | 0.1164 | 0.0025 | 2% | 0.1825 | 0.0020 | 1% | 0.2134 | 0.0048 | 2% | 0.0920 | 0.0012 | 1% |
| 28 | 0.1254 | 0.0009 | 1% | 0.1935 | 0.0007 | 0% | 0.1820 | 0.0035 | 2% | 0.1753 | 0.0022 | 1% |
| 29 | 0.1135 | 0.0027 | 2% | 0.2047 | 0.0039 | 2% | 0.1868 | 0.0003 | 0% | 0.1662 | 0.0008 | 1% |
| 30 | 0.1250 | 0.0022 | 2% | 0.2077 | 0.0011 | 1% | 0.1704 | 0.0031 | 2% | 0.1800 | 0.0030 | 2% |

Table 2. shows the normalized OD (450 nm) for triplicate readings of duplicate wells from each control and sample. Data underlined were considered outliers (box-plot approach) for the dataset and not included in the analysis for the cut point calculations.

TABLE 2

| | A Day 1 | | | A Day 2 | | | B Day 1 | | | B day 2 | | |
|---|---|---|---|---|---|---|---|---|---|---|---|---|
| | Mean | SD | CV | Mean | SD | CV | Mean | SD | CV | Mean | SD | CV |
| NBP | 0.7765 | 0.1793 | 23% | 0.8508 | 0.1052 | 12% | 0.5043 | 0.1334 | 26% | 0.4078 | 0.0395 | 10% |
| LPC | 0.5833 | 0.0134 | 2% | 0.5024 | 0.0075 | 1% | 0.4226 | 0.0027 | 1% | 0.2543 | 0.0070 | 3% |
| MPC | 0.4953 | 0.0042 | 1% | 0.3046 | 0.0129 | 4% | 0.1671 | 0.0074 | 4% | 0.1651 | 0.0049 | 3% |
| HPC | 0.2043 | 0.0078 | 4% | 0.1536 | 0.0129 | 8% | 0.1394 | 0.0085 | 6% | 0.1362 | 0.0040 | 3% |
| US | 0.1528 | 0.0087 | 6% | 0.1272 | 0.0044 | 3% | 0.1197 | 0.0122 | 10% | 0.1425 | 0.0016 | 1% |
| 1 | 0.9884 | 0.0310 | 3% | 1.1504 | 0.0170 | 1% | 0.4036 | 0.0021 | 1% | 0.4217 | 0.0074 | 2% |
| 2 | 1.0914 | 0.0106 | 1% | 1.3661 | 0.0304 | 2% | 0.5771 | 0.0049 | 1% | 0.6719 | 0.0300 | 4% |
| 3 | 1.0799 | 0.0612 | 6% | 1.3206 | 0.0642 | 5% | 0.5151 | 0.0050 | 1% | 0.5261 | 0.0076 | 1% |
| 4 | 1.0392 | 0.0877 | 8% | 1.3405 | 0.0144 | 1% | 0.6108 | 0.0155 | 3% | 0.6053 | 0.0177 | 3% |
| 5 | 1.1224 | 0.0359 | 3% | 1.3417 | 0.0063 | 0% | 0.6692 | 0.0081 | 1% | 0.5430 | 0.0176 | 3% |
| 6 | 1.0447 | 0.0398 | 4% | 1.2815 | 0.0201 | 2% | 0.6703 | 0.0215 | 3% | 0.5793 | 0.0018 | 0% |
| 7 | 1.0973 | 0.0199 | 2% | 1.2522 | 0.0274 | 2% | 0.5941 | 0.0245 | 4% | 0.5449 | 0.0165 | 3% |
| 8 | 1.0893 | 0.0305 | 3% | 1.4318 | 0.0377 | 3% | 0.5395 | 0.0015 | 0% | 0.5555 | 0.0118 | 2% |
| 9 | 1.1279 | 0.0206 | 2% | 1.2242 | 0.0241 | 2% | 0.5977 | 0.0137 | 2% | 0.4775 | 0.0176 | 4% |
| 10 | 1.1449 | 0.0302 | 3% | 1.3652 | 0.0063 | 0% | 0.5558 | 0.0138 | 2% | 0.4883 | 0.0103 | 2% |
| 11 | 0.9162 | 0.0321 | 4% | 1.1368 | 0.0563 | 5% | 0.9989 | 0.0088 | 1% | 0.6469 | 0.0228 | 4% |
| 12 | 0.9264 | 0.0067 | 1% | 1.0858 | 0.0032 | 0% | 0.5214 | 0.0123 | 2% | 0.4898 | 0.0142 | 3% |
| 13 | 0.9178 | 0.0203 | 2% | 1.2469 | 0.0138 | 1% | 1.0272 | 0.0225 | 2% | 0.5857 | 0.0173 | 3% |
| 14 | 0.9002 | 0.0377 | 4% | 1.1365 | 0.0246 | 2% | 0.6545 | 0.0098 | 2% | 0.5165 | 0.0182 | 4% |
| 15 | 0.9381 | 0.0320 | 3% | 1.2624 | 0.0887 | 7% | 0.7926 | 0.0157 | 2% | 0.6504 | 0.0052 | 1% |
| 16 | 1.0309 | 0.0134 | 1% | 1.1069 | 0.0469 | 4% | 0.6515 | 0.0170 | 3% | 0.4285 | 0.0060 | 1% |
| 17 | 1.1034 | 0.0059 | 1% | 1.1516 | 0.0174 | 2% | 0.7270 | 0.0278 | 4% | 0.4499 | 0.0140 | 3% |
| 18 | 1.0940 | 0.0447 | 4% | 1.0959 | 0.0083 | 1% | 0.4403 | 0.0064 | 1% | 0.5137 | 0.0139 | 3% |
| 19 | 1.1008 | 0.0296 | 3% | 1.2606 | 0.0153 | 1% | 0.7797 | 0.0153 | 2% | 0.4326 | 0.0113 | 3% |
| 20 | 0.9949 | 0.0118 | 1% | 1.0217 | 0.0167 | 2% | 0.4614 | 0.0086 | 2% | 0.6209 | 0.0171 | 3% |
| 21 | 1.0614 | 0.0277 | 3% | 1.0047 | 0.0194 | 2% | 0.7683 | 0.0117 | 2% | 0.5023 | 0.0127 | 3% |
| 22 | 0.9966 | 0.0119 | 1% | 0.9459 | 0.0178 | 2% | 0.6658 | 0.0124 | 2% | 0.3836 | 0.0167 | 4% |
| 23 | 0.9890 | 0.0170 | 2% | 1.0053 | 0.0409 | 4% | 0.7405 | 0.0102 | 1% | 0.4537 | 0.0162 | 4% |
| 24 | 0.9146 | 0.0121 | 1% | 1.0175 | 0.0085 | 1% | 0.7320 | 0.0118 | 2% | 0.4280 | 0.0057 | 1% |
| 25 | 0.8855 | 0.0026 | 0% | 0.9061 | 0.0407 | 4% | 0.5273 | 0.0151 | 3% | 0.4104 | 0.0142 | 3% |
| 26 | 0.7956 | 0.0032 | 0% | 1.1274 | 0.0312 | 3% | 0.5002 | 0.0145 | 3% | 0.4267 | 0.0082 | 2% |
| 27 | 0.8292 | 0.0311 | 4% | 1.0527 | 0.0148 | 1% | 0.7112 | 0.0283 | 4% | 0.3399 | 0.0114 | 3% |
| 28 | 0.9252 | 0.0347 | 4% | 1.0820 | 0.0250 | 2% | 0.6199 | 0.0134 | 2% | 0.6929 | 0.0170 | 2% |
| 29 | 0.8312 | 0.0388 | 5% | 1.0891 | 0.0235 | 2% | 0.6322 | 0.0179 | 3% | 0.6361 | 0.0199 | 3% |
| 30 | 0.9208 | 0.0317 | 3% | 1.0898 | 0.0482 | 4% | 0.6140 | 0.0426 | 7% | 0.6746 | 0.0268 | 4% |

For the calculation of the cut point, outliers were eliminated using the outlier box-plot approach that identifies points above the 75th percentile plus 1.5 times the interquartile range (high outliers) and below the 25th percentile minus 1.5 times the interquartile range (low outliers). Normality of the datasets was assessed using the Kolmogorov-Smirnov test. All the datasets except the phosphorylated receptor concentration data for analyst A on day 1 passed the normality test after outliers were eliminated (Table 3). Statistical differences of the assay means and homogeneity of variances were assessed on the log transformed datasets using an ANOVA test treating the assay runs as a fixed effect. The sets of normalized data (both for OD and receptor concentration) failed the equal variance test. The means of the assay runs were determined to be significantly different indicating the need of a floating cut point for the assay (Table 4). The Normality and Equal Variance tests indicate that the log-transformed OD data should be used in the calculation of the cut point.

TABLE 3
Normality test results for the four
data sets (after removing outliers).

| | | Normalized | | Norm. |
|---|---|---|---|---|
| Data Set | OD | OD | [P-PDGF-Rb] | [P-PDGF-Rb] |
| A; Day 1 | K-S dist = | K-S dist = | K-S dist = | K-S dist = |
| | 0.157 | 0.109 | 0.161 | 0.098 |
| | p = 0.057 | p > 0.193 | p = 0.045 | p > 0.200 |
| | n = 30 | n = 30 | n = 30 | n = 30 |
| A; Day 2 | K-S dist = | K-S dist = | K-S dist = | K-S dist = |
| | 0.093 | 0.132 | 0.108 | 0.120 |
| | p > 0.200 | p = 0.193 | p > 0.200 | p > 0.200 |
| | n = 30 | n = 30 | n = 30 | n = 30 |
| B; Day 1 | K-S dist = | K-S dist = | K-S dist = | K-S dist = |
| | 0.089 | 0.092 | 0.088 | 0.103 |
| | p > 0.200 | p > 0.200 | p > 0.200 | p > 0.200 |
| | n = 28 | n = 28 | n = 28 | n = 28 |
| B; Day 2 | K-S dist = | K-S dist = | K-S dist = | K-S dist = |
| | 0.109 | 0.099 | 0.113 | 0.118 |
| | p > 0.200 | p > 0.200 | p > 0.200 | p > 0.200 |
| | n = 26 | n = 30 | n = 26 | n = 28 |

TABLE 4
Comparison of assay means across the four
assay runs using log-transformed data.

| | | Normalized | | Norm. |
|---|---|---|---|---|
| Data Set | OD | OD | [P-PDGF-Rb] | [P-PDGF-Rb] |
| Normality | Passed | Passed | Passed | Passed |
| | p = 0.364 | p = 0.469 | p = 0.084 | p < 0.134 |
| Equal | Passed | Failed | Passed | Failed |
| Variance | p = 0.404 | p < 0.050 | p = 0.199 | p < 0.050 |
| Differences | Yes | Yes | Yes | Yes |
| between | p < 0.001 | p < 0.001 | p < 0.001 | p < 0.001 |
| groups | | | | |

Cut points for each assay run were calculated using the log-transformed OD data according to parametric, robust parametric and empirical methods with an allowance of 1% false positives (99^{th} percentile). For the parametric approach, the cut point was calculated as the mean minus 2.33 times the standard deviation (SD); for the robust parametric approach it was calculated as the median minus 2.33 times 1.483 times the median absolute deviation (MAD); the empiric approach determines the 99th percentile of the data (Table 5).

TABLE 5
Cut point calculations using the log-transformed OD values.

| | | | Robust | | | |
|---|---|---|---|---|---|---|
| | Parametric | | Parametric | | | |
| | Mean − | | Median − (2.33 × | | Empiric | |
| | (2.33 × SD) | | (1.483 × MAD | | 99^{th} Percentile | |
| Data | Cut | Cut Point | Cut | Cut | Cut | Cut Point |
| Set | Point | OD | Point | Point | Point | OD |
| A; Day 1 | −0.9899 | 0.1024 | −1.0187 | 0.0958 | −0.9620 | 0.1091 |
| A; Day 2 | −0.8030 | 0.1574 | −0.8302 | 0.1479 | −0.7699 | 0.1699 |
| B; Day 1 | −0.8860 | 0.1300 | −0.8940 | 0.1276 | −0.8613 | 0.1376 |
| B; Day 2 | −1.0519 | 0.0887 | −1.0685 | 0.0854 | −1.0280 | 0.0938 |
| A | −1.0494 | 0.0893 | −1.1617 | 0.0689 | −0.9549 | 0.1110 |
| B | −1.1014 | 0.0792 | −1.2275 | 0.0592 | −1.0186 | 0.0958 |
| Pooled | −1.0791 | 0.0834 | −1.1885 | 0.0648 | −1.0021 | 0.0995 |
| Data | | | | | | |

Determination of the normalization factor for the calculation of the cut point was performed using the log-transformed OD values for the negative base pool of each assay run and the corresponding robust parametric cut point (Table 6). The cut point of the assay should be calculated using the geometric mean of OD values for the negative base pool and a multiplicative normalization factor of 0.8511.

TABLE 6
Robust parametric normalization factor.

| | | Robust | | OD |
|---|---|---|---|---|
| | Negative | Parametric | Additive | Multiplicative |
| | Base | Cut | Normalization | Normalization |
| Data Set | Pool | Point | Factor | Factor |
| A; Day 1 | −0.9651 | −1.0187 | −0.0536 | 0.8839 |
| A; Day 2 | −0.7805 | −0.8302 | −0.0497 | 0.8919 |
| B; Day 1 | −0.7978 | −0.8940 | −0.0962 | 0.8013 |
| B; Day 2 | −0.9860 | −1.0685 | −0.0824 | 0.8271 |
| A | — | — | −0.0516 | 0.8879 |
| B | — | — | −0.0893 | 0.8142 |
| Pooled | — | — | −0.0705 | 0.8511 |
| Data | | | | |

Using the normalization factor determined above, cut points were calculated for each of the 4 assay runs. In all cases, the mean OD for the negative base pool (NBP) was determined to be negative for the presence of anti-rhPDGF-BB neutralizing antibodies and all three positive controls (LPC, MPC, HPC) were determined positive for the presence of anti-rhPDGF-BB neutralizing activity. No phosphorylation of the receptor was observed in the unstimulated control (Table 7). Values in italics are negative for anti-rhPDGF-BB neutralizing antibodies, and values in bold are positive for anti-rhPDGF-BB neutralizing antibodies.

TABLE 7
Cut points and controls for each assay run.

| Data Set | Cut Point | NBP | LPC | MPC | HPC | US |
|---|---|---|---|---|---|---|
| A; Day 1 | 0.0922 | 0.1084 | 0.0799 | 0.0703 | 0.01314 | 0.0228 |
| A; Day 2 | 0.1411 | 0.1658 | 0.1017 | 0.0662 | 0.0316 | 0.0276 |
| B; Day 1 | 0.13568 | 0.1593 | 0.1306 | 0.0603 | 0.0455 | 0.0414 |
| B; Day 2 | 0.08979 | 0.1033 | 0.0667 | 0.0441 | 0.0362 | 0.0345 |

### Precision

The levels of rhPDGF-BB-induced receptor phosphorylation in MG-63 cell lysates were measured after treatment with the assay controls: negative base pool (NBP), high positive control (HPC), medium positive control (MPC), and low positive control (LPC). Each of two analysts (A and C) performed the assay three times in three different days with 6 sets of controls each day for a total of 12 assay runs and 36 sets of controls. The intra-assay (Tables 8 and 9), inter-assay (Tables 10 and 11), and inter-analyst (Table 12) coefficients of variation (CV) were all under the pre-specified 30%.

TABLE 8
Average OD (450 nm) for triplicate readings from six replicate sets of
assay controls by analyst C in the Phospho-PDGF Rβ Elisa (intra-assay precision):

| | | 1 | 2 | 3 | 4 | 5 | 6 | Mean | S.D. | C.V. |
|---|---|---|---|---|---|---|---|---|---|---|
| Day 1 | NBP | 0.1696 | 0.1707 | 0.1660 | 0.1894 | 0.1606 | 0.2061 | 0.1771 | 0.0172 | 9.7% |
| | LPC | 0.1276 | 0.1449 | 0.1182 | 0.1322 | 0.1373 | 0.1480 | 0.1347 | 0.0111 | 8.2% |
| | MPC | 0.0743 | 0.0836 | 0.0831 | 0.0755 | 0.1032 | 0.0722 | 0.0820 | 0.0114 | 13.9% |
| | HPC | 0.0397 | 0.0348 | 0.0368 | 0.0408 | 0.0399 | 0.0358 | 0.0380 | 0.0025 | 6.5% |
| Day 2 | NBP | 0.1146 | 0.1226 | 0.1378 | 0.1338 | 0.1393 | 0.1414 | 0.1316 | 0.0107 | 8.1% |
| | LPC | 0.0774 | 0.0838 | 0.0737 | 0.0962 | 0.0937 | 0.0742 | 0.0832 | 0.0098 | 11.8% |
| | MPC | 0.0472 | 0.0537 | 0.0613 | 0.0520 | 0.0563 | 0.0530 | 0.0539 | 0.0047 | 8.7% |
| | HPC | 0.0316 | 0.0301 | 0.0325 | 0.0318 | 0.0317 | 0.0319 | 0.0316 | 0.0008 | 2.6% |
| Day 3 | NBP | 0.1422 | 0.1420 | 0.1588 | 0.1519 | 0.1503 | 0.1780 | 0.1539 | 0.0134 | 8.7% |
| | LPC | 0.1438 | 0.1221 | 0.1103 | 0.1156 | 0.1426 | 0.1138 | 0.1247 | 0.0148 | 11.9% |
| | MPC | 0.1069 | 0.0843 | 0.0719 | 0.0923 | 0.0997 | 0.1033 | 0.0931 | 0.0132 | 14.2% |
| | HPC | 0.0319 | 0.0287 | 0.0401 | 0.0303 | 0.0302 | 0.0296 | 0.0318 | 0.0042 | 13.1% |

TABLE 9
Average OD (450 nm) for triplicate readings from six replicate sets of
assay controls by analyst A in the Phospho-PDGF Rβ Elisa (intra-assay precision):

| | | 1 | 2 | 3 | 4 | 5 | 6 | Mean | S.D. | C.V. |
|---|---|---|---|---|---|---|---|---|---|---|
| Day 1 | NBP | 0.1478 | 0.1580 | 0.1649 | 0.1465 | 0.1702 | 0.1712 | 0.1598 | 0.0108 | 6.8% |
| | LPC | 0.1391 | 0.1292 | 0.1335 | 0.1305 | 0.1283 | 0.1279 | 0.1314 | 0.0043 | 3.2% |
| | MPC | 0.0863 | 0.0844 | 0.0895 | 0.0849 | 0.0780 | 0.0661 | 0.0815 | 0.0084 | 10.4% |
| | HPC | 0.0401 | 0.0365 | 0.0396 | 0.0371 | 0.0374 | 0.0393 | 0.0383 | 0.0015 | 4.0% |
| Day 2 | NBP | 0.1749 | 0.1934 | 0.2195 | 0.1961 | 0.2211 | 0.2265 | 0.2053 | 0.0202 | 9.9% |
| | LPC | 0.1404 | 0.1428 | 0.1580 | 0.1460 | 0.1624 | 0.1456 | 0.1492 | 0.0089 | 5.9% |
| | MPC | 0.0856 | 0.0800 | 0.0955 | 0.0812 | 0.0800 | 0.0646 | 0.0811 | 0.0100 | 12.4% |
| | HPC | 0.0351 | 0.0283 | 0.0317 | 0.0302 | 0.0324 | 0.0303 | 0.0314 | 0.0023 | 7.4% |
| Day 3 | NBP | 0.1487 | 0.1310 | 0.1545 | 0.1376 | 0.1456 | 0.1639 | 0.1469 | 0.0118 | 8.0% |
| | LPC | 0.1052 | 0.1153 | 0.1112 | 0.1123 | 0.1187 | 0.0951 | 0.1096 | 0.0084 | 7.7% |
| | MPC | 0.0539 | 0.0582 | 0.0552 | 0.0568 | 0.0577 | 0.0587 | 0.0567 | 0.0019 | 3.3% |
| | HPC | 0.0306 | 0.0307 | 0.0307 | 0.0324 | 0.0293 | 0.0299 | 0.0306 | 0.0010 | 3.4% |

TABLE 10
Average OD (450 nm) for 18 replicate sets of assay controls
analyzed by analyst C over the course of three days in
the Phospho-PDGF Rβ Elisa (intra-assay precision):

| | Mean | S.D. | C.V. |
|---|---|---|---|
| NBP | 0.1542 | 0.0232 | 15.0% |
| LPC | 0.1142 | 0.0256 | 22.4% |
| MPC | 0.0763 | 0.0196 | 25.7% |
| HPC | 0.0338 | 0.0041 | 12.0% |

TABLE 11
Average OD (450 nm) for 18 replicate sets of assay controls
analyzed by analyst A over the course of three days in
the Phospho-PDGF Rβ Elisa (intra-assay precision):

| | Mean | S.D. | C.V. |
|---|---|---|---|
| NBP | 0.1706 | 0.0293 | 17.2% |
| LPC | 0.1301 | 0.0181 | 13.9% |
| MPC | 0.0731 | 0.0139 | 19.0% |
| HPC | 0.0334 | 0.0039 | 11.7% |

TABLE 12
Average OD (450 nm) for 36 replicate sets of assay controls
by both analysts over the course of three days in the
Phospho-PDGF Rβ Elisa (intra-assay precision):

| | Mean | S.D. | C.V. |
|---|---|---|---|
| NBP | 0.1624 | 0.0274 | 16.8% |
| LPC | 0.1221 | 0.0233 | 19.1% |
| MPC | 0.0747 | 0.0168 | 22.5% |
| HPC | 0.0336 | 0.0039 | 11.7% |

### Sensitivity.

The levels of receptor phosphorylation in MG-63 cell lysates were measured after treatment with rhPDGF-BB pre-incubated with a neutralizing anti-PDGF-BB antibody in pooled human serum. The assay was performed by three analysts; each analyst performed the assay three times in three different days with two series of dilutions of the neutralizing anti-PDGF-BB antibody each day. The cut point for each assay plate was calculated as described above using the mean OD values for the samples without added neutralizing antibodies (Tables 13-15). The dose/response curves were fitted to a 4-parameter logistic model in the antibody concentration range of 2,000.0 to 15.6 ng/mL. These models were used to calculate the concentration of antibody corresponding to the cut point. The sensitivity of the assay was calculated using different confidence levels using the t-distribution of antibody concentrations corresponding to the cut points (Table 16).

TABLE 13
Average OD (450 nm) for triplicate readings from each antibody
dose/response series by Analyst B (two series per day):

| | 1.1 | 1.2 | 2.1 | 2.2 | 3.1 | 3.2 | 4.1 | 4.2. |
|---|---|---|---|---|---|---|---|---|
| 4,000.0 | 0.0356 | 0.0320 | 0.0324 | 0.0302 | 0.0335 | 0.0300 | 0.0315 | 0.0280 |
| 2,000.0 | 0.0373 | 0.0342 | 0.0318 | 0.0305 | 0.0315 | 0.0288 | 0.0293 | 0.0306 |
| 1,000.0 | 0.0451 | 0.0425 | 0.0307 | 0.0308 | 0.0301 | 0.0290 | 0.0291 | 0.0291 |
| 500.0 | 0.0627 | 0.0659 | 0.0336 | 0.0360 | 0.0371 | 0.0365 | 0.0360 | 0.0372 |
| 250.0 | 0.0850 | 0.1015 | 0.0497 | 0.0484 | 0.0527 | 0.0601 | 0.0547 | 0.0592 |
| 125.0 | 0.1227 | 0.1274 | 0.0813 | 0.0732 | 0.0838 | 0.0852 | 0.0738 | 0.0760 |
| 62.5 | 0.1322 | 0.1101 | 0.1017 | 0.0790 | 0.1252 | 0.1042 | 0.0842 | 0.0944 |
| 31.3 | 0.0984 | 0.0985 | 0.1147 | 0.1237 | 0.1038 | 0.1114 | 0.0984 | 0.1034 |
| 15.6 | 0.1158 | 0.1096 | 0.1247 | 0.1293 | 0.1047 | 0.1055 | 0.1033 | 0.0972 |
| 7.8 | 0.1058 | 0.1349 | 0.1377 | 0.1426 | 0.1215 | 0.0998 | 0.1134 | 0.1050 |
| 0.0 | 0.1234 | 0.1395 | 0.1161 | 0.1728 | 0.0914 | 0.1018 | 0.0984 | 0.0984 |
| Cut Point | 0.1050 | 0.1187 | 0.0988 | 0.1471 | 0.0778 | 0.0867 | 0.0837 | 0.0837 |

TABLE 14
Average OD (450 nm) for triplicate readings from each antibody
dose/response series by Analyst A (two series per day):

| | 1.1 | 1.2 | 2.1 | 2.2 | 3.1 | 3.2 |
|---|---|---|---|---|---|---|
| 4,000.0 | 0.0263 | 0.0239 | 0.0244 | 0.0208 | 0.0237 | 0.0222 |
| 2,000.0 | 0.0247 | 0.0237 | 0.0198 | 0.0196 | 0.0245 | 0.0219 |
| 1,000.0 | 0.0324 | 0.0285 | 0.0207 | 0.0200 | 0.0235 | 0.0232 |
| 500.0 | 0.0832 | 0.0634 | 0.0322 | 0.0322 | 0.0305 | 0.0295 |
| 250.0 | 0.1256 | 0.1112 | 0.0695 | 0.0713 | 0.0664 | 0.0616 |
| 125.0 | 0.1918 | 0.1233 | 0.0971 | 0.1023 | 0.1114 | 0.1029 |
| 62.5 | 0.1992 | 0.1510 | 0.1171 | 0.1047 | 0.1384 | 0.1189 |
| 31.3 | 0.1803 | 0.1722 | 0.1422 | 0.1295 | 0.1359 | 0.1179 |
| 15.6 | 0.2037 | 0.1791 | 0.1586 | 0.1605 | 0.1578 | 0.1268 |
| 7.8 | 0.1900 | 0.1865 | 0.1631 | 0.1716 | 0.1707 | 0.1354 |
| 0.0 | 0.1878 | 0.1676 | 0.1654 | 0.1676 | 0.1731 | 0.1640 |
| Cut Point | 0.1598 | 0.1427 | 0.1408 | 0.1426 | 0.1474 | 0.1396 |

TABLE 15
Average OD (450 nm) for triplicate readings from each antibody
dose/response series by Analyst C (two series per day):

| | 1.1 | 1.2 | 2.1 | 2.2 | 3.1 | 3.2 |
|---|---|---|---|---|---|---|
| 4,000.0 | 0.0396 | 0.0370 | 0.0362 | 0.0342 | 0.0442 | 0.0375 |
| 2,000.0 | 0.0373 | 0.0377 | 0.0340 | 0.0348 | 0.0400 | 0.0370 |
| 1,000.0 | 0.0362 | 0.0396 | 0.0352 | 0.0380 | 0.0412 | 0.0382 |
| 500.0 | 0.0387 | 0.0402 | 0.0543 | 0.0550 | 0.0554 | 0.0597 |
| 250.0 | 0.0522 | 0.0576 | 0.0791 | 0.0921 | 0.0851 | 0.0845 |
| 125.0 | 0.0710 | 0.0770 | 0.0894 | 0.0927 | 0.1001 | 0.0952 |
| 62.5 | 0.0949 | 0.1000 | 0.1097 | 0.1000 | 0.1120 | 0.0932 |
| 31.3 | 0.0958 | 0.1066 | 0.1247 | 0.1011 | 0.1190 | 0.1101 |
| 15.6 | 0.0958 | 0.1044 | 0.1050 | 0.1179 | 0.1244 | 0.1182 |
| 7.8 | 0.1003 | 0.1094 | 0.1226 | 0.1024 | 0.1085 | 0.1166 |
| 0.0 | 0.1090 | 0.1266 | 0.1265 | 0.1213 | 0.1402 | 0.1128 |
| Cut Point | 0.0927 | 0.1078 | 0.1076 | 0.1032 | 0.1193 | 0.0960 |

TABLE 16
Calculated sensitivities (ng/mL) using different data ranges for
interpolation of the cut point ODs and different confidence levels.

| | | | | Sensitivity | | Sensitivity | | Sensitivity |
|---|---|---|---|---|---|---|---|---|
| | | | | (99% | | (95% | | (90% |
| | Mean | S.D. | t_{0.01} | confidence) | t_{0.05} | confidence) | t_{0.05} | confidence) |
| 4,000.0-0.0 | 81.05 | 57.69 | 2.86 | 246.08 | 2.09 | 201.78 | 1.73 | 180.79 |
| 2,000.0-0.0 | 83.35 | 54.68 | 2.86 | 239.78 | 2.09 | 197.79 | 1.73 | 177.89 |
| 2,000-7.8 | 82.48 | 54.17 | 2.92 | 240.69 | 2.12 | 197.31 | 1.75 | 177.05 |
| 2,000-15.6 | 75.96 | 48.26 | 2.95 | 218.18 | 2.13 | 178.83 | 1.75 | 160.57 |
| 1,000-0.0 | 77.55 | 55.50 | 2.86 | 236.34 | 2.09 | 193.71 | 1.73 | 173.52 |
| 1,000-7.8 | 81.19 | 55.13 | 2.92 | 242.23 | 2.12 | 198.07 | 1.75 | 177.45 |
| 1,000-15.6 | 81.41 | 54.78 | 2.92 | 241.41 | 2.12 | 197.57 | 1.75 | 177.05 |

The assay sensitivity, with 99% confidence, is approximately 220 ng/mL of goat anti-PDGF-BB antibody; this is the antibody used as positive control in the assay. The assay sensitivity, with 95% confidence, is approximately 180 ng/mL of goat anti-PDGF-BB antibody and 160 ng/mL of goat anti-PDGF-BB antibody with 90% confidence. These calculations likely overestimate the sensitivity due to the high variability of the data. This variability is, in part, due to the fact that the control antibody is polyclonal and the dose/response curves become very “noisy” when the antibody is at low concentrations.

### System Suitability and Acceptable OD Ranges

The execution of the experiments for assessment of the precision of the assay provided an opportunity to gather a large amount of data for the controls that will be used in the assay. The combined efforts of both analysts over several days compiled 36 sets of data for each of the 4 controls and 6 sets of data for standard curves of the assay. The standards provided with the ELISA kits will be used as a verification of system suitability. Tables 17 and 18 below summarize these data and the ranges of acceptable ODs for each of these samples (controls and standards) calculated as the mean of the data ±3 times the standard deviation.

TABLE 17
Mean OD values for 36 replicate sets of controls
and acceptable mean OD value ranges calculated from
these data in the phosphorylated PDGF Rβ ELISA.

| | Mean | S.D. | C.V. | Range |
|---|---|---|---|---|
| NBP | 0.1542 | 0.0232 | 15.0% | 0.0803-0.2445 |
| LPC | 0.1142 | 0.0256 | 22.4% | 0.0523-0.1920 |
| MPC | 0.0763 | 0.0196 | 25.7% | 0.0243-0.1252 |
| HPC | 0.0338 | 0.0041 | 12.0% | 0.0218-0.0454 |

TABLE 18
Mean OD values for 6 replicate sets of phosphorylated
PDGF Rβ standards (pg/mL) and acceptable mean
OD value ranges calculated from these data.

| | Mean | S.D. | C.V. | Range |
|---|---|---|---|---|
| 500.00 | 1.1289 | 0.2169 | 19% | 0.4782-1.7796 |
| 250.00 | 0.6317 | 0.1242 | 20% | 0.2590-1.0044 |
| 125.00 | 0.3586 | 0.0672 | 19% | 0.1570-0.5601 |
| 62.50 | 0.2043 | 0.0346 | 17% | 0.1005-0.3081 |
| 31.25 | 0.1230 | 0.0188 | 15% | 0.0667-0.1793 |
| 15.63 | 0.0816 | 0.0123 | 15% | 0.0446-0.1186 |
| 7.81 | 0.0621 | 0.0067 | 11% | 0.0420-0.0823 |
| 0.00 | 0.0387 | 0.0051 | 13% | 0.0234-0.0541 |

All references to singular characteristics or limitations of the present disclosure shall include the corresponding plural characteristic or limitation, and vice versa, unless otherwise specified or clearly implied to the contrary by the context in which the reference is made. It should be understood that the forgoing related only to certain embodiments of the present disclosure and that numerous modifications or alterations may be made therein without departing from the spirit and scope of the present disclosure.

All combinations of method or process steps as used herein can be performed in any order, unless otherwise specified or clearly implied to the contrary by the context in which the referenced combination is made.

The methods and compositions of the present disclosure, including components thereof, can comprise, consist of, or consist essentially of the essential elements and limitations of the embodiments described herein, as well as any additional or optional ingredients, components or limitations described herein.

As used herein, the term “about” should be construed to refer to both of the numbers specified in any range. Any reference to a range should be considered as providing support for any subset within that range.

All patents, publications and abstracts cited above are incorporated herein by reference in their entirety.

What is claimed is:

1. A method for detecting the presence of growth factor neutralizing antibodies in a serum sample, comprising:
contacting a population of cells with i) a serum sample, and ii) the growth factor, wherein the cells comprise a growth factor receptor;
detecting an amount of a biomarker in the population of cells, wherein the biomarker is indicative of binding of the growth factor to the growth factor receptor, and
correlating the amount of the biomarker with the presence of the growth factor neutralizing antibodies.

2. The method of claim 1, wherein the growth factor is selected from the group consisting of platelet-derived growth factor (PDGF), bone morphogenic proteins (BMPs), epidermal growth factor (EGF), fibroblast growth factor (FGF), insulin-like growth factor (IGF), transforming growth factor-α (TGF-α), transforming growth factor-β (TGFβ), tumor necrosis factor-α (TNF-α), and vascular endothelial growth factor (VEGF).

3. The method of claim 1 or 2, wherein the biomarker is a phosphorylated growth factor receptor.

4. The method of any one of claims 1 to 3, wherein the phosphorylated growth factor receptor is detected using an enzyme-linked immunosorbent assay (ELISA) assay.

5. The method of claim 4, wherein the phosphorylated growth factor receptor is detected by measuring an optical density.

6. The method of any one of claims 1 to 5, wherein the contacting step comprises incubating the cells with the serum sample.

7. The method of any one of claims 1 to 6, wherein the serum sample is preincubated with the growth factor prior to the contacting step.

8. The method of any one of claims 1 to 7, wherein the cells are lysed prior to the detecting step.

9. The method of any one of claims 1 to 8, wherein the cells are human cells.

10. The method of any one of claims 1 to 9, wherein the cells are human neonatal fibroblast cells.

11. The method of any one of claims 1 to 10, wherein the cells are MG-63 osteosarcoma cells.

12. The method of claim 11, further comprising serum-starving the MG-63 osteosarcoma cells for a period of time prior to the contacting step.

13. The method of claim 12, wherein the period of time ranges from about 4 hours to about 48 hours, about 4 hours to about 24 hours, about 4 hours to about 16 hours, about 4 hours to about 12 hours, or about 6 hours to about 12 hours.

14. The method of any one of claims 1 to 13, wherein the serum sample is taken from a subject who has received a treatment comprising the growth factor.

15. The method of any one of claims 1 to 14, wherein the method is performed in vitro.

16. The method of any one of claims 1-15, further comprising:
determining a floating cut point based on a negative base pool,
correlating the floating cut point with the presence of growth factor neutralizing antibodies, and
comparing the amount of the biomarker in the population of cells to the floating cut point.

17. The method of claim 16, wherein the floating cut point is determined by:
contacting a second population of cells with i) a negative base pool sample, and the growth factor, wherein the cells comprise a growth factor receptor;
detecting an amount of a phosphorylated growth factor receptor in the second population of cells.

18. The method of claim 16 or 17, wherein the floating cut point is tied to a statistical measure of the negative base pool.

19. The method of claim 18, wherein the statistical measure is a standard deviation, standard error, a mean, a median, a median absolute deviation, or a fit parameter.

20. The method of claim 19, further comprising assigning a multiplicative factor to determine the floating cut point.

21. The method of any one of claims 16 to 20, wherein a detected amount of the biomarker in the serum sample greater than about 80% of the floating cut point indicates that the serum sample does not contain growth factor neutralizing antibodies.

22. The method of any one of claims 1 to 21, wherein the growth factor is PDGF.

23. The method of claim 22, wherein the PDGF is selected from the group consisting of PDGF-AA, PDGF-BB, PDGF-AB, PDGF-CC, PDGF-DD and combinations thereof.

24. The method of claim 23, wherein the PDGF is PDGF-BB.

25. The method of any one of claims 23 to 24, wherein the PDGF is recombinant human (rh) PDGF.

26. The method of claim 25, wherein the rhPDGF is selected from the group consisting of rhPDGF-AA, rhPDGF-BB, rhPDGF-AB, rhPDGF-CC, rhPDGF-DD and combinations thereof.

27. The method of claim 26, wherein the PDGF is rhPDGF-BB.

28. The method of any one of claims 22 to 27, wherein the growth factor neutralizing antibodies are anti-PDGF-BB antibodies.

29. A method of determining the presence of growth factor neutralizing antibodies in a subject who has received a treatment comprising PDGF, comprising:
providing a serum sample from the subject,
contacting a population of cells with i) a serum sample, and ii) the growth factor, wherein the cells comprise a growth factor receptor;
detecting an amount of a biomarker in the population of cells, wherein the biomarker is indicative of binding of the growth factor to the growth factor receptor, and
correlating the amount of the biomarker with the presence of the growth factor neutralizing antibodies.

30. The method of claim 28, further comprising discontinuing treatment in the subject with treatment comprising the growth factor when growth factor neutralizing antibodies are detected in the subject.


SEQUENCE LISTING (6 sequences)

SEQ ID NO: 1 - DNA, 30 bp, Artificial Sequence
  Synthetic NanE forward Primer 1
gcggccgcaccatgtcgttacttgcacaac

SEQ ID NO: 2 - DNA, 29 bp, Artificial Sequence
  Synthetic NanE Reverse Primer 1
atgcggccgctcatagcaccgcctttttc

SEQ ID NO: 3 - DNA, 19 bp, Artificial Sequence
  Synthetic NanE Forward Primer 2
gcggccgcaccatgtcgtt

SEQ ID NO: 4 - DNA, 19 bp, Artificial Sequence
  Synthetic NanE Reverse Primer 2
atgcggccgctcatagcac

SEQ ID NO: 5 - DNA, 711 bp, Escherichia coli
  misc_feature (1)..(11): Synthetic region of amplified PCR product having a NotI recognition site flanking 5′ end of ORF encoding E. coli GlcNAc-6-P 2′ epimerase
gcggccgcaccatgtcgttacttgcacaattggatcaaaaaatcgctgctaacggtggcctgattgtctcctgccagccggttccggacagcccgctcgataaacccgaaatcgtcgccgccatggcattagcggcagaacaggcgggcgcggttgccattcgcattgaaggtgtggcaaatctgcaagccacgcgtgcggtggtgagcgtgccgattattggaattgtgaaacgcgatctggaggattctccggtacgcatcacggcctatattgaagatgttgatgcgctggcgcaggcgggcgcggacattatcgccattgacggcaccgaccgcccgcgtccggtgcctgttgaaacgctgctggcacgtattcaccatcacggtttactggcgatgaccgactgctcaacgccggaagacggcctggcatgccaaaagctgggagccgaaattattggcactacgctttctggctataccacgcctgaaacgccagaagagccggatctggcgctggtgaaaacgttgagcgacgccggatgtcgggtgattgccgaagggcgttacaacacgcctgctcaggcggcggatgcgatgcgccacggcgcgtgggcggtgacggtcggttctgcaatcacgcgtcttgagcacatttgtcagtggtacaacacagcgatgaaaaaggcggtgctatgagcggccgcat

SEQ ID NO: 6 - protein, 229 aa, Escherichia coli
MSLLAQLDQKIAANGGLIVSCQPVPDSPLDKPEIVAAMALAAEQAGAVAIRIEGVANLQATRAVVSVPIIGIVKRDLEDSPVRITAYIEDVDALAQAGADIIAIDGTDRPRPVPVETLLARIHHHGLLAMTDCSTPEDGLACQKLGAEIIGTTLSGYTTPETPEEPDLALVKTLSDAGCRVIAEGRYNTPAQAADAMRHGAWAVTVGSAITRLEHICQWYNTAMKKAVL